(19) United States
(12) Patent Application Publication
(10) Pub. No.: US 2024/0018454 A1
(43) Pub. Date: Jan. 18, 2024

(54) MICRO-ORGANOSPHERES FOR USE IN PERSONALIZED MEDICINE AND DRUG DEVELOPMENT

(71) Applicants: Daniel DELUBAC, Durham, NC (US); Daniel NELSON, Durham, NC (US); John CALAWAY, Durham, NC (US); Daniel FREED, Druham, NC (US); Xilis, Inc., Druham, NC (US)

(72) Inventors: Daniel DELUBAC, Leesburg, VA (US); Daniel NELSON, Holly Springs, NC (US); John CALAWAY, Pittsboro, NC (US); Daniel FREED, New Hill, NC (US)

(21) Appl. No.: 18/038,854

(22) PCT Filed: Nov. 23, 2021
(86) PCT No.: PCT/US2021/060525

§ 371 Date: May 25, 2023

Related U.S. Application Data
(60) Provisional application No. 63/118,527, filed on Nov. 25, 2020.

(51) Int. Cl. C12M 3/06; C12M 1/34; B01L 3/00

(52) U.S. Cl. CPC C12M 23/16; C12M 41/36; C12M 41/12; B01L 3/502761; B01L 2300/0867; B01L 2200/0652; B01L 2400/043

(57) ABSTRACT

Disclosed herein are systems, apparatuses, and methods for forming micro-organospheres. In some variations, a system may comprise a micro-organosphere generator configured to form a set of micro-organospheres from a mixture of a biological sample and a fluid. A controller may be coupled to an imaging device. The controller may be configured to receive the imaging data corresponding to one or more of the mixture or the set of micro-organospheres, and estimate one or more characteristics of the set of micro-organospheres based at least on the imaging data.

### CROSS-REFERENCE TO RELATED APPLICATIONS

This application claims priority to U.S. Provisional Application Ser. No. 63/118,527, filed on Nov. 25, 2020, the contents of which are hereby incorporated by reference in its entirety.

### TECHNICAL FIELD

The disclosure relates generally to personalized medicine and drug development, and in particular to generating micro-organospheres and using them in personalized medicine and drug development.

### BACKGROUND

Model cell and tissue systems are useful for biological and medical research. The most common practice is to derive immortalized cell lines from tissue and culture them in two-dimensional (2D) conditions (e.g., in a Petri dish or well plate). Although useful for basic research, 2D cell lines do not correlate well with individual patient response to therapy. Three-dimensional (3D) cell culture models are proving particularly helpful in developmental biology, disease pathology, regenerative medicine, drug toxicity and efficacy testing, and personalized medicine. For example, spheroids and organoids are 3D cell aggregates that have been studied. However, both organoids and spheroids have limitations that reduce their efficacy.

Organoids are in vitro derived cell aggregates that include a population of stem cells that can differentiate into cells of major cell lineages. Organoids typically have a diameter of more than one mm. They typically grow and expand more slowly than 2D cell culture. To generate organoids from clinical samples, the input sample must contain hundreds of thousands of viable cells; so organoids often cannot be made from low volume samples such as from a biopsy; and when they can be made, they must be cultured for several weeks before being ready for experimental use. Organoids are also highly variable in size, shape and cell number. As such, additional 3D tissue model systems, devices, and methods may be desirable.

### SUMMARY

The present disclosure relates generally to systems and methods for forming micro-organospheres. In one aspect, the disclosure provides a system comprising a micro-organosphere generator comprising a microfluidic device and configured to form a set of micro-organospheres from a mixture of a biological sample and a fluid. A controller may be coupled to the imaging device. The controller may be configured to receive imaging data corresponding to one or more of the mixture or the set of micro-organospheres, and estimate one or more characteristics of the set of micro-organospheres based at least on the imaging data.

In some variations, an imaging device may be configured to generate the imaging data corresponding to the one or more of the mixture or the set of micro-organospheres. In some variations, a cell culture vessel may be coupled to the imaging device and configured to culture the set of micro-organospheres in a plurality of wells. The controller may be configured to estimate a number of micro-organospheres in the plurality of wells based at least on the imaging data. In some variations, a cell culture vessel may be coupled to the imaging device and configured to culture the set of micro-organospheres in a plurality of wells. The controller may be configured to estimate a number of micro-organospheres in the plurality of wells based at least on the imaging data.

In some variations, one or more sensors may be coupled to the microfluidic device and configured to generate sensor data corresponding to the mixture or the set of micro-organospheres. The controller may be configured to receive the sensor data from the one or more sensors, and estimate one or more characteristics of the set of micro-organospheres based at least on the sensor data. In some variations, one or more pumps may be coupled to the microfluidic device and configured to control fluid flow to the microfluidic device. A temperature regulator may be coupled to the microfluidic device, sample source, or fluid source, and configured to control a temperature of the sample source, the fluid source, the mixture, or the set of micro-organospheres. The controller may be configured to modify one or more of the pump or the temperature based at least on the imaging data and the sensor data.

In some variations, a polymerizer may be fluidically coupled to the microfluidic device and configured to polymerize the mixture to form the set of micro-organospheres. In some variations, a demulsifier may be fluidically coupled to the microfluidic device and configured to demulsify the mixture to form the set of micro-organospheres. In some variations, the demulsifier may comprise a flow separator configured to isolate the set of micro-organospheres. In some variations, the flow separator may extend along a length of the demulsifier. In some variations, an agitator may be configured to agitate the micro-organospheres within a fluid at a predetermined concentration.

In some variations, the one or more of the characteristics of the set of micro-organospheres may comprise one or more of a micro-organosphere diameter, a total number of cells, or a number of living cells. In some variations, the controller may be configured to estimate one or more characteristics of the mixture based at least on the imaging data. In some variations, the one or more of the characteristics of the mixture may comprise a total number of cells and a number of living cells.

In some variations, the imaging data corresponds to the biological sample, and the controller may be configured to estimate one or more characteristics of the biological sample based at least on the imaging data. In some variations, the one or more of the characteristics of the biological sample may comprise a total number of cells and a number of living cells. In some variations, the set of micro-organospheres may comprise a diameter of between about 200 μm and about 400 μm.

In some variations, the micro-organosphere generator may be configured to form the set of micro-organospheres from the biological sample comprising a volume of up to about 1 mL. In some variations, the micro-organosphere generator may be configured to form the set of micro-organospheres from the biological sample comprising less than about 10,000 cells. In some variations, the biological sample may comprise between about 3,500 cells and about 7,500 cells.

In some variations, the micro-organosphere generator may be configured to form the set of micro-organospheres from the biological sample having a volume of about 5 μL to about 5 mL. In some variations, the biological sample may have a volume of about 5 about 10 about μL, about 35.3 μL, about 50 μL, about 100 μL, about 250 about 500 about 1 mL, about 1.5 mL, about 2 mL, about 2.5 mL, about 3 mL, about 3.5 mL, about 4 mL, about 4.5 mL, or about 5 mL.

In some variations, the set of micro-organospheres may comprise a set of non-cellular objects. In some variations, the set of non-cellular objects may comprise one or more inert particles. In some variations, the set of non-cellular objects may comprise between about 1 inert particle and about 5,000 inert particles.

Another aspect of the present disclosure relates to a system comprising a micro-organosphere generator configured to form a set of micro-organospheres from a mixture of a biological sample and a fluid, and a controller configured to receive imaging data corresponding to the set of micro-organospheres, and identify the set of micro-organospheres comprising a diameter of between about 50 μm and about 500 μm based at least on the imaging data.

In some variations, an imaging device may be configured to generate the imaging data corresponding to the set of micro-organospheres. In some variations, the biological sample corresponds to a patient biopsy.

Another aspect of the present disclosure relates to a method of making a micro-organosphere composition in a system, comprising providing the biological sample comprising dissociated cells and an unpolymerized base material, forming the mixture from the biological sample in an immiscible solution, and polymerizing the mixture to form a set of micro-organospheres.

In some variations, the biological sample may be dissociated to obtain the dissociated cells. In some variations, the base material may be temperature sensitive and polymerization occurs when the temperature of the mixture is increased. In some variations, the set of micro-organospheres may comprise a mean diameter of between about 50 μm and about 500 μm with a coefficient of variability (CV) less than about 30% CV, less than about 20% CV, or less than about 10% CV.

In some variations, the organospheres may be sorted by size to form the set of micro-organospheres comprising a mean diameter of between about 50 μm and about 500 μm with a coefficient of variability (CV) less than about 30% CV, less than about 20% CV, or less than about 10% CV, or one or more flow rates may be controlled within the micro-organosphere generator to form the set of micro-organospheres comprising a mean diameter of between about 50 μm and about 500 μm with a coefficient of variability (CV) less than about 30% CV, less than about 20% CV, or less than about 10% CV.

In some variations, an assay may be performed on the micro-organospheres to determine treatment response. In some variations, the assay may be a cell viability assay or a cell painting assay. In some variations, the assay may be performed in 14 days or less from when the biological sample is obtained from a patient. In some variations, the micro-organospheres may comprise between about 1 dissociated primary cell and about 1,000 dissociated primary cells distributed within the base material. In some variations, the biological sample may correspond to a patient biopsy.

Another aspect of the present disclosure relates to a micro-organosphere composition comprising a plurality of micro-organospheres with each micro-organosphere including a base material and at least one organoid. The plurality of micro-organospheres may comprise parameters comprising a predetermined number of cells per droplet, a predetermined number of droplets in the composition, and/or a predetermined droplet size. Each of the parameters may independently comprise a coefficient of variability (CV) less than about 30% CV, less than about 20% CV, or less than about 10% CV.

In some variations, the mean diameter of each micro-organosphere in the composition may be between about 50 μm and about 500 μm. In some variations, the mean diameter of each micro-organosphere in the composition may comprise a coefficient of variability (CV) of less than about 30% CV, less than about 20% CV, or less than about 10% CV.

In some variations, each micro-organosphere may comprise a base material and only one organoid. In some variations, each micro-organosphere may comprise an inert particle. In some variations, the inert particle may be a magnetic particle, a magnetizable particle, a fluorescent particle, or a combination thereof. In some variations, each micro-organosphere may comprise between about 1 inert particle and about 5,000 inert particles.

In some variations, the plurality of micro-organospheres may comprise tissue from a patient biopsy. In some variations, the tissue may comprise non-cultured cells. In some variations, the micro-organospheres may comprise between about 1 dissociated primary cell and about 1,000 dissociated primary cells distributed within the base material.

Another aspect of the present disclosure relates to a method of immobilizing micro-organospheres in a well or culture plate, the method comprising providing a plurality of micro-organospheres, each micro-organosphere comprising a base material, at least one organoid, and a magnetic or magnetizable particle, and applying a magnetic field to the well or culture plate, thereby immobilizing the micro-organospheres to a surface of the well or culture plate.

In some variations, the well or the culture plate has a bottom, and the micro-organospheres are immobilized to the bottom of the well or culture plate.

Another aspect of the present disclosure relates to a method of immobilizing micro-organospheres in a well or culture plate that has a bottom, the method comprising providing a plurality of micro-organospheres, each micro-organosphere comprising a base material and at least one organoid, functionalizing the bottom with an antibody that binds the base material, and contacting the micro-organospheres with the antibody, thereby immobilizing the micro-organospheres to the bottom.

In some variations, the antibody may be immobilized on the bottom by incubation. In some variations, the bottom may be coated with protein A and/or protein G prior to the functionalization.

Another aspect of the present disclosure relates to a method of determining a patient's response to a treatment, the method comprising performing an assay on micro-organospheres, wherein the micro-organospheres are produced by mixing a biological sample comprising dissociated cells from the patient with an unpolymerized base material in an immiscible solution to produce a mixture, and polymerizing the mixture to form a set of micro-organospheres.

In some variations, the assay may be a cell viability assay or a cell painting assay. In some variations, the assay may be performed in about 14 days or less from when the biological sample is obtained from a patient. In some variations, the micro-organospheres may comprise between about 1 dissociated primary cell and about 1,000 dissociated primary cells distributed within the base material.

Additional variations, features, and advantages of the invention will be apparent from the following detailed description and through practice of the invention.

### BRIEF DESCRIPTION OF THE DRAWINGS

FIG. 1 is a block diagram of an illustrative variation of a micro-organosphere forming system.

FIG. 2A is a schematic diagram of an illustrative variation of a micro-organosphere forming system. FIG. 2B is a schematic diagram of an illustrative variation of a demulsifier. FIG. 2C is a schematic diagram of another illustrative variation of a micro-organosphere forming system. FIG. 2D is a schematic diagram of another illustrative variation of a micro-organosphere forming system.

FIG. 3A is a schematic diagram of an illustrative variation of a micro-organosphere forming system. FIG. 3B is a schematic diagram of another illustrative variation of a micro-organosphere forming system.

FIG. 4A is a plan view of an illustrative variation of a micro-organosphere forming system. FIG. 4B is a perspective view of the micro-organosphere forming system depicted in FIG. 4A in a closed configuration. FIG. 4C is a perspective view of the micro-organosphere forming system depicted in FIG. 4A in an open configuration.

FIG. 5 is a cross-sectional view of an illustrative variation of a micro-organosphere generator.

FIG. 6A is a schematic diagram of an illustrative variation of a demulsifier. FIG. 6B is a cross-sectional view of an illustrative variation of a demulsifier.

FIG. 7 is an image of an illustrative variation of a micro-organosphere forming system.

FIG. 8 is a flowchart of an illustrative variation of a method of forming micro-organospheres.

FIG. 9 is a flowchart of another illustrative variation of a method of forming micro-organospheres.

FIG. 10 is a block diagram of an illustrative variation of a method of estimating a biological sample and a mixture. QC refers to quality control.

FIG. 11 is a block diagram of an illustrative variation of a method of estimating micro-organospheres.

FIG. 12 is a block diagram of an illustrative variation of a method of outputting micro-organospheres.

FIG. 13 is a block diagram of another illustrative variation of a method of estimating micro-organospheres.

FIG. 14 is an image generated by an illustrative variation of an imaging device.

FIG. 15A are images of an illustrative variation of micro-organospheres comprising organoids at various stages of development.

FIG. 15B is a plot of an illustrative variation of organoid development within micro-organospheres.

FIG. 16 are images of an illustrative variation comparing a breast cancer micro-organosphere at day 3 to a conventional organoid at day 3.

FIG. 17 is a schematic diagram of an illustrative variation of producing micro-organospheres comprising non-cellular objects.

FIGS. 18A and 18B are graphs of illustrative variations depicting a derivatization of culture plates with mouse anti-laminin and/or mouse anti-collagen IV antibodies via either direct binding to polystyrene (FIG. 18A) or protein A-mediated attachment (FIG. 18B).

FIG. 19 is a schematic diagram of illustrative variations of producing micro-organospheres.

### DETAILED DESCRIPTION

Systems and methods for forming micro-organospheres (e.g., droplets, droplet micro-organospheres (DMOS)) are described herein. In some variations, drug compositions may be screened using micro-organospheres to predict effective therapies that may be applied to a patient. For example, a toxicity screen for drugs or other chemical compositions may be performed based on micro-organospheres comprising healthy tissue and/or cancerous (e.g., tumor) tissue from a patient.

In some variations, micro-organospheres may be configured to encapsulate one or more living cells, including but not limited to, cancer cells, stromal cells, cell lines, combinations thereof, and the like, for culture. For example, the systems and methods may generate micro-organospheres having a predetermined size or size distribution with a predetermined number of cells, and a predetermined concentration.

In some variations, a set of micro-organospheres may be formed from patient-derived tumor samples that have been dissociated and suspended in a basement matrix (e.g., Matrigel®). In some variations, the micro-organospheres may be patterned onto a microfluidic microwell array to be incubated and dosed with one or more drug compounds. This miniaturized assay may enable efficient drug screening from a small tumor sample.

As used herein, the term “micro-organosphere” may refer to a droplet formed from a solid or semi-solid base material that contains cells cultured to form organoid(s) where the droplet has a diameter of between about 50 μm and about 500 μm, between about 50 μm and about 400 μm, between about 50 μm and about 300 μm, and between about 50 μm and about 250 μm, including all values and sub-ranges in-between. In some variations, the base material can include an extracellular matrix (e.g., a hydrogel such as Matrigel®). The micro-organosphere can include one, two, three, four, five, or more organoids. In some variations, micro-organospheres may initially comprise between about 1 and about 1,000 dissociated primary cells distributed within the base material, between about 1 and about 750, between about 1 and about 500, between about 1 and about 400, between about 1 and about 300, between about 1 and about 200, between about 1 and about 150, between about 1 and about 100, between about 1 and about 75, between about 1 and about 50, between about 1 and about 40, between about 1 and about 30, and between about 1 and about 20, including all values and sub-ranges in-between. In some variations, the micro-organosphere further comprises an inert particle. In some variations, the inert particle is a magnetic particle, a magnetizable particle, a fluorescent particle, or a combination thereof.

In some variations, a system may optionally comprise a micro-organosphere generator configured to form a set of micro-organospheres from a mixture of a biological sample and a fluid. An imaging device may be configured to generate imaging data corresponding to the set of micro-organospheres. A controller (e.g., processor and memory) may be coupled to the imaging device, and the controller may be configured to receive the imaging data from the imaging device, and identify the set of micro-organospheres having diameter(s) within a predetermined range (e.g., between about 50 μm and about 500 μm) based on the imaging data and/or other sensor data. For example, one or more characteristics of the set of micro-organospheres may be estimated based at least on the imaging data.

The systems and methods of forming micro-organospheres described herein may increase one or more of speed, throughput, consistency, or heterogeneity. By contrast, conventional organoids are in vitro derived cell aggregates that typically have a diameter of more than about 1 mm diameter, and have a large amount of variability in organoid size, shape and number of cells. They also require large numbers of viable cells (e.g., hundreds of thousands) and take extended periods of time (e.g., month) to culture and expand.

### I. System

Overview

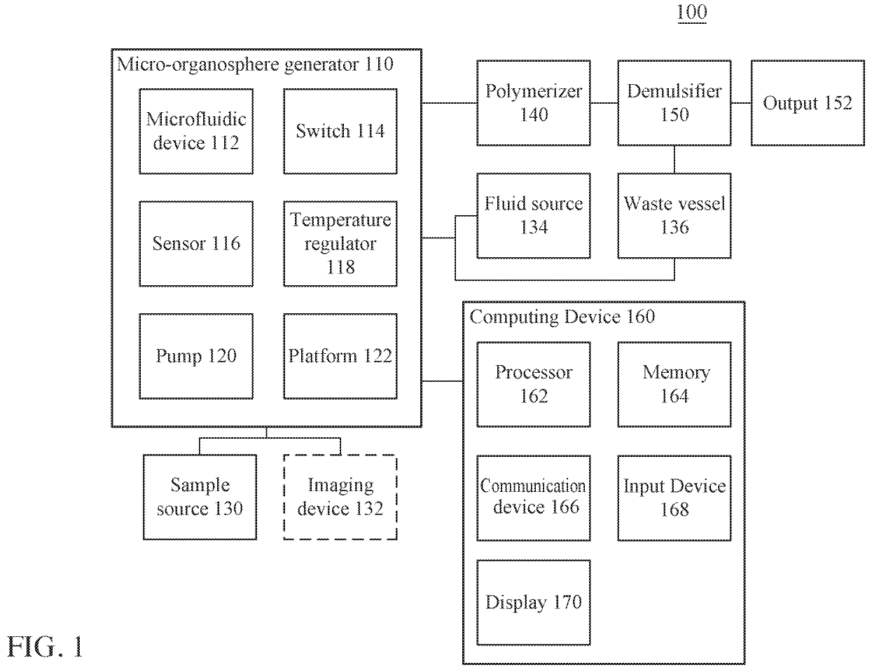

Described here are systems and apparatuses configured to form micro-organospheres. In some variations, micro-organospheres may be formed based on predetermined criteria (e.g., size, number, density). Micro-organosphere formation may include one or more steps of generating, polymerizing, and demulsifying. FIG. 1 is a block diagram of a micro-organosphere forming system 100 comprising a micro-organosphere generator 110, a sample source 130, an optional imaging device 132, a fluid source 134, a waste vessel 136, a polymerizer 140, a demulsifier 150, an output 152, and a computing device 160. In some variations, a micro-organosphere generator 110 may comprise one or more of a microfluidic device 112 (e.g., microfluidic chip), a switch 114, a sensor 116, a temperature regulator 118, a pump 120, or a platform 122. In some variations, the computing device 160 may comprise a processor 162, a memory 164, a communication device 166, an input device 168, and a display 170 (e.g., output device).

The systems described herein provide numerous advantages over conventional organoid production methods. For example, the cells in the micro-organospheres generated by the micro-organosphere system 100 may establish and grow faster than cells seeded in conventional organoids. In some variations, the micro-organospheres described herein may be generated with high throughput (e.g., millions per hour) and the systems may be compatible with other high-throughput screening devices. Furthermore, the number of droplets seeded per well may be controlled in a predetermined manner. For example, the systems described herein may be compatible with one or more components of a robotic liquid handling system by controlling a droplet size and ensuring that the droplets are smaller than the bore size of pipette tips or a channel diameter of existing technologies. Components of robotic liquid handling systems generally include microplate dispensers, liquid handlers, and multi-well plates (e.g., 24-well plates, 48-well plates, 96-well plates, 1536-well plates). The systems described herein can also be compatible with other automation instruments such as a vacuum, a plate washer, a centrifuge, an incubator, an imager, a microscope, a plate reader, a sealer, and a peeler.

In some variations, micro-organospheres may be established at a higher rate than conventional organoids. For example, the local environment inside a micro-organosphere may facilitate the exchange of growth factors, nutrients, and other components in culture media (e.g., growth media) to promote growth of organoids, whereas conventional organoid domes result in nutrient and growth factor gradients that dramatically affect the biology of organoids depending on their relative position within the dome (e.g., in the center versus in the periphery). The result is a lower likelihood that dissociated tumor stem cells will encounter an environment optimized for their proliferation and re-acquisition of a tumor-like structure. In some variations, the micro-organosphere environment may offer a homogeneous microenvironment optimized for diffusion of key nutrients, which increases the establishment success rate.

The micro-organospheres described herein may also be more heterogeneous than conventional organoids. The volume of a micro-organosphere constrains each original cell (e.g., tumor cell) in a smaller volume than conventional organoids. As such, clonal takeover by rapidly dividing cells is constrained by the size (e.g., diameter) of the micro-organosphere. This characteristic of micro-organospheres may facilitate the analysis of biologically and clinically relevant subclones. Although this sequestration may be lost over multiple passages, individual micro-organospheres may be recovered and cultured separately, allowing for the isolation of specific subclones. The ability to separate distinct clonal populations also facilitates studies aimed at understanding molecular factors contributing to drug sensitivity and resistance. In some variations, imaging may be used to identify and isolate one or more distinct subclones.

Cells grown in a micro-organosphere may acquire a 3D structure more representative of the source tissue or tumor more reliably and faster than with conventional organoids. For example, the local environment inside a droplet may facilitate exchange of growth factors, nutrients, and other components in the culture media which promote growth of organoids. Facilitated diffusion of nutrients throughout relatively small, spherical droplets may result in a higher propensity for establishment on a faster timescale.

Micro-organospheres and apparatuses for forming thereof are described in International Patent Application No. PCT/US2020/026275, and titled “METHODS AND APPARATUSES FOR PATIENT-DERIVED MICRO-ORGANOSPHERES,” the entire disclosure of which is incorporated herein by reference in its entirety.

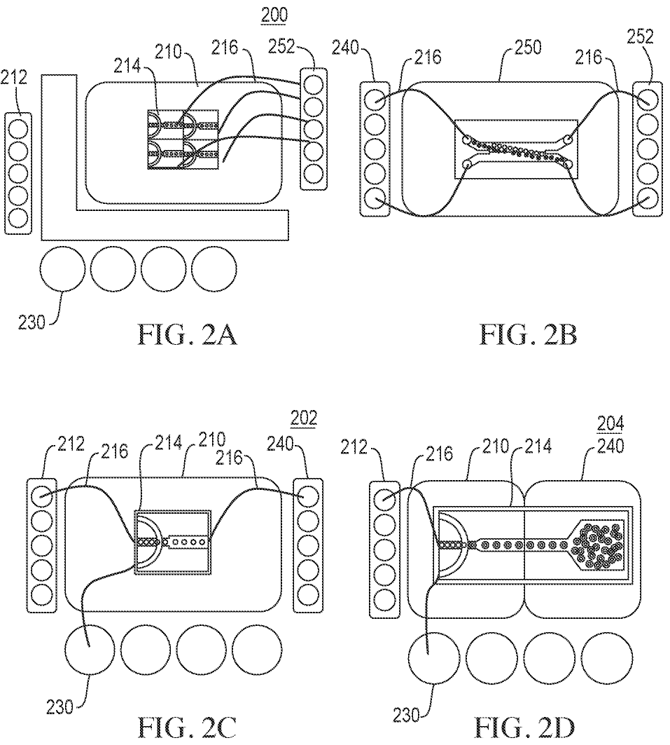

FIG. 2A is a schematic diagram of a micro-organosphere forming system 200 comprising one or more of a micro-organosphere generator 210, a sample source 212, a fluid source 230, an output 252, or one or more fluid conduits 216 configured to be in fluidic communication between the output 252 and the micro-organosphere generator 210. The micro-organosphere generator 210 may comprise a plurality of microfluidic devices 214 configured to manufacture micro-organospheres simultaneously (e.g., in parallel operation). In some variations, the fluid source 230 may comprise a bulk oil and/or a cleaning fluid. In some variations, the output 252 may include a plurality of recovery vessels configured to separately receive respective outputs of the plurality of microfluidic devices 214.

FIG. 2B is a schematic diagram of a demulsifier 250 fluidically coupled between a polymerizer 240 and an output 252. For example, the demulsifier 250 may be fluidically coupled to an output of the polymerizer 240 and the output 252 may be fluidically coupled to an output of the demulsifier 250 using respective fluid conduits 216. In some variations, the polymerizer 240 and the demulsifier 250 may be temperature regulated at about 37° C.

FIG. 2C is a schematic diagram of a micro-organosphere forming system 202 comprising a micro-organosphere generator 210, a sample source 212, a fluid source 230, and a polymerizer 240. In some variations, the micro-organosphere generator 210 may comprise a microfluidic device 214. The microfluidic device 214 may be fluidically coupled to a sample source 212, a fluid source 230, and a polymerizer 240 using respective fluid conduits 216. For example, the microfluidic device 214 may receive an input from the sample source 212 and the fluid source 230, and output one or more micro-organospheres to the polymerizer 240. In FIG. 2C, the micro-organosphere generator 210 and the polymerizer 240 are separated from each other.

FIG. 2D is a schematic diagram of a micro-organosphere forming system 204 comprising a micro-organosphere generator 210, a sample source 212, a fluid source 230, and a polymerizer 240. In some variations, the micro-organosphere generator 210 may comprise a microfluidic device 214. The microfluidic device 214 may be fluidically coupled to a sample source 212, a fluid source 230, and a polymerizer 240 using respective fluid conduits 216. For example, the microfluidic device 214 may receive an input from the sample source 212 and the fluid source 230, and output a micro-organosphere to the polymerizer 240. In FIG. 2D, the micro-organosphere generator 210 and the polymerizer 240 may be coupled together.

In some variations, one or more of the systems 200, 202, 204, and demulsifier 250 may be pressure and temperature controlled (e.g., regulated). In some variations, one or more portions of the microfluidic devices 214 may be visualized (e.g., viewable by a user, imaged by an imaging device). For example, a junction (e.g., intersection, T-junction) of a microfluidic device may be visible for imaging. In some variations, one or more of the microfluidic devices 214 may be sterilized (e.g., washed) at predetermined intervals (e.g., after each run).

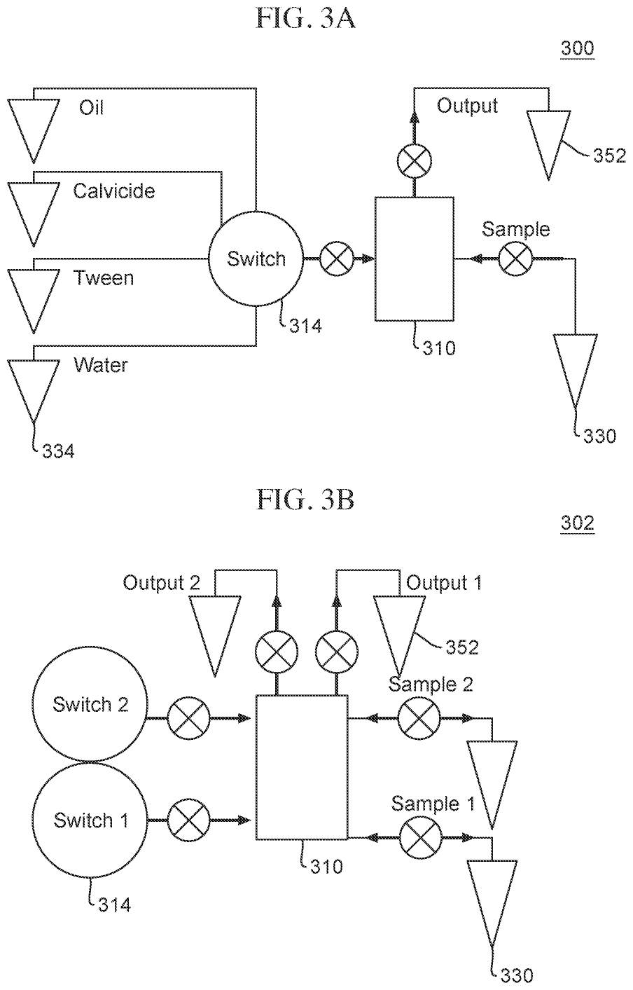

FIG. 3A is a schematic diagram of a micro-organosphere forming system 300 comprising a micro-organosphere generator 310, a switch 314, a sample source 330, a fluid source 334, and an output 352. In some variations, the system 300 may comprise a single-channel configuration where the micro-organosphere generator 310 comprises a single channel for each of the sample source 330, fluid source 334, and output 352.

FIG. 3B is a schematic diagram of a micro-organosphere forming system 302 comprising a micro-organosphere generator 310, a switch 314, a sample source 330, a fluid source 334, and an output 352. In some variations, the system 302 may comprise a multi-channel configuration where the micro-organosphere generator 310 comprises a plurality of channels for each of the sample source 330, fluid source 334, and output 352. The multi-channel configuration may allow flexibility, reduce run times, and promote continuous operation, as well as cleaning (e.g., washing, sterilization) between runs.

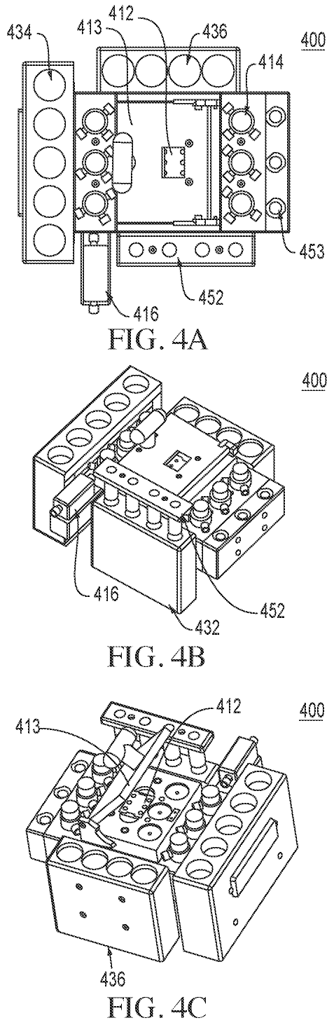

FIG. 4A is a plan view of a micro-organosphere forming system 400 comprising a microfluidic device 412, a cover 413, a switch 414 (e.g., embedded switches), a sensor 416 (e.g., output flow sensor), a fluid source 434 (e.g., 50 mL bulk reagents), a waste vessel 436 (e.g., 50 mL waste module), an output 452 (e.g., 1.7 mL output adapter), and a reservoir 453 (e.g., reduced sample reservoir). FIG. 4B is a perspective view of the micro-organosphere forming system 400 in a closed configuration (e.g., where the cover is 413 is closed over the microfluidic device 412) and FIG. 4C is a perspective view of the micro-organosphere forming system 400 depicted in an open configuration (e.g., where the cover is 413 is opened to facilitate access to the microfluidic device 412). In some variations, a micro-organosphere generation process may be performed when the cover 413 is in the closed configuration. In some variations, the open configuration facilitates operator access to the microfluidic device 412. In some variations, the cover 413 may comprise a transparent portion configured to enable visual access to the microfluidic device 412 (e.g., for an imaging device).

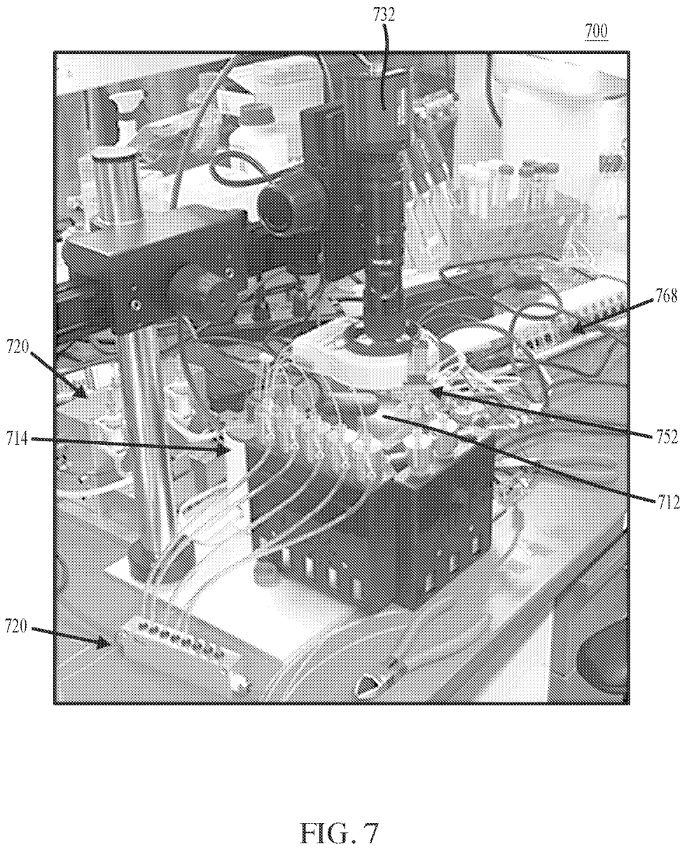

FIG. 7 is an image of an illustrative variation of a micro-organosphere forming system 700 comprising a microfluidic device 712, a switch 714, a pump 720 (e.g., fluid pump, air pump), an imaging device 732, an output 752 (e.g., output line), and an input device 768 (e.g., switch control).

Micro-Organosphere Generator

In some variations, a micro-organosphere generator 110 (e.g., DMO S, generator, droplet generator) may comprise at least a partially enclosed enclosure (e.g., housing) in which one or more automated micro-organosphere forming steps are performed. For example, the micro-organosphere generator 110 may be configured to transfer a sample source 130 and a fluid source 134 into a microfluidic device 112 using one or more switches 114, pumps 120, and platforms 122. The temperature regulator 118 and pump 120 may be configured to facilitate micro-organosphere 110 formation in the microfluidic device 112. Optionally, one or more sensors 116 and/or imaging devices 132 may be configured to monitor the micro-organosphere formation process. The computing device 160 may be configured to receive sensor data and/or imaging data and used to control the micro-organosphere generator 110. One or more fluid conduits (e.g., connectors, tubes, connectors, lines) may be in fluid communication between the microfluidic devices 114 and the pump 120, sample source 130, fluid source 134, and/or waste vessel 136.

In some variations, the micro-organosphere generator 110 may comprise transparent windows and/or openings to enable visual access to the micro-organosphere generation process (e.g., sample, fluid, mixture, micro-organosphere).

Microfluidic Device

In some variations, the micro-organosphere generator 110 may comprise one or more microfluidic devices 112 fluidically coupled to one or more of a sample source 130 or a fluid source 134. In some variations, micro-organospheres may be formed from a single microfluidic device 114 using a sample volume of about 5 μL to about 5 mL, including all ranges and sub-values in-between. In some variations, the sample volume can be about 5 μL, about 10 μL, about 20 μL, about 35.3 μL, about 50 μL, about 100 μL, about 250 μL, and about 500 μL, about 1 mL, about 1.5 mL, about 2 mL, about 2.5 mL, about 3 mL, about 3.5 mL, about 4 mL, about 4.5 mL, or about 5 mL.

In some variations, the sample may include less than about 10,000 cells to about 100,000 cells, including all ranges and sub-values in-between. In some variations, the sample may include about 3,500 to about 100,000 cells. In some variations, the sample may include about 3,500 to about 7,500 cells.

In some variations, the formed micro-organospheres may comprise a concentration of about 10 cells per 17 nL micro-organosphere, about 20 cells per 17 nL micro-organosphere, or about 100 cells per 17 nL micro-organosphere, including all ranges and sub-values in-between.

In some variations, the micro-organosphere generator 110 may comprise a plurality of microfluidic devices 112 operated simultaneously, enabling higher-throughput parallel operation. In some variations, the microfluidic device 112 may comprise a releasable cover configured to allow cleaning and reuse of the device 112. For example, one or more fluidic channels and a microfluidic device 114 may be sterilized (e.g., washed) for reuse. In some variations, the microfluidic device 112 may comprise a transparent portion configured for visual access and to enable imaging as described in more detail herein.

In some variations, a biological sample may be prepared in a biosafety cabinet and enclosed (e.g., sealed) within a microfluidic device 112, thereby preventing contamination. In some variations, the microfluidic device 112 may be used with the micro-organosphere generator 110. In some variations, the micro-fluidic device 112 may be configured for single-use (e.g., as a single-use consumable).

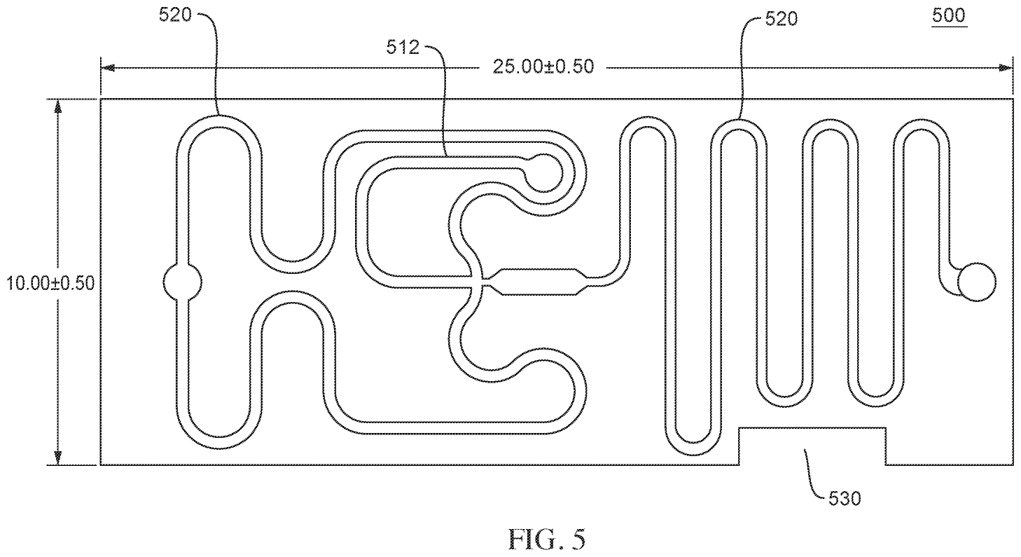

FIG. 5 is a cross-sectional view of an illustrative variation of a microfluidic device 500 comprising an input channel 512, an output channel 520, and an engagement feature 530. In some variations, the engagement feature (e.g., a notch) can be configured to fit into a corresponding micro-organosphere system (e.g., housing) in a predetermined configuration. That is, the engagement feature may ensure that the microfluidic device 500 is loaded in a predetermined orientation and is not loaded otherwise. In some variations, the microfluidic device 500 may be configured to generate micro-organospheres comprising a diameter of about 300 μm. In some variations, the channels 512, 520 may comprise a serpentine shape configured to minimize device 500 size while maintaining laminar flow with a predetermined back pressure. In some variations, larger diameter portions of the channels 512, 520 may be configured to prevent primary samples (e.g., cellular clumps, protein aggregates, debris) from clogging the microfluidic device 500. The microfluidic device 500 may be a single-use or multi-use device.

Switch

In some variations, the micro-organosphere generator 110 may comprise one or more switches 114 (e.g., valves) coupled to a microfluidic device 112, a sample source 130, a fluid source 134, and/or a waste vessel 136 and configured to provide input/output control to the microfluidic device 112 and ensure consistent processing of sample sources 130 with repeatable output metrics. In some variations, the switches 114 may be controlled by the computing device 160 and may operate in response to, for example, sensor data generated by sensor 116 and image data generated by imaging device 132.

Sensor

In some variations, the micro-organosphere generator 110 may comprise one or more sensors 116 configured to monitor one or more components and/or steps of a micro-organosphere forming process. In some variations, the one or more sensors 116 may comprise one or more optical sensors, mechanical sensors, voltage and/or resistance (or capacitance, or inductance) sensors, force sensors, combinations thereof, and the like. In some variations, one or more sensors 116 may be configured to measure one or more parameters such as flow, pressure, pH, dissolved gas concentration, osmolality, turbidity, hydration, conductivity, absorbance, nutrient concentration, waste concentration, ion concentration, oxygen concentration, temperature, combinations thereof, and the like.

In some variations, a flow sensor coupled to the microfluidic device 112 may be configured to generate sensor data (e.g., flow rate data) which may be received by the computing device 160 to control one or more of the micro-organosphere generator 110 (e.g., switch 114, temperature regulator 118, pump 120), polymerizer 140, or demulsifier 150. For example, an extracellular matrix such as Matrigel® may comprise a wide range of viscosity when used to form a micro-organosphere, which may result in a flow rate change at a constant pressure. In some variations, a flow sensor may be configured to measure flow rate in the microfluidic device 112. A consistent flow rate may be maintained by varying the pressure (e.g., via pump 120) in response to the measured flow rate, thereby improving the consistency of droplet formation. In some variations, pressure and temperature may be controlled based on one or more of sensor data and/or imaging data.

In some variations, one or more sensors (e.g., proximity sensors) may be configured to measure a position of a generator 110 enclosure (e.g., open cover, closed cover). That is, separate portions of a proximity sensor may be positioned on a side of a cover (e.g., lid) and generate a signal corresponding to an open state and a closure state.

Temperature Regulator

In some variations, the micro-organosphere generator 110 may comprise one or more temperature regulators 120. The temperature regulator 120 may comprise one or more of a heater, a cooler (e.g., Peltier device), or a temperature sensor coupled to one or more of a micro-organosphere generator 110 (e.g., microfluidic device 112, switch 114, pump 120, fluid conduits), a sample source 130, a fluid source 134, a polymerizer 140, or a demulsifier 150. In some variations, the computing device 160 may couple to and be configured to control the temperature regulator 118 based on, for example, sensor data and/or imaging data. In some variations, the temperature regulator 118 may be configured to polymerize temperature activated hydrogels (e.g., Matrigel®).

Pump

In some variations, the micro-organosphere generator 110 may comprise one or more pumps 120 (e.g., fluid pumps) configured to control fluid flow into and out of the microfluidic devices 112. In some variations, one or more pumps may be coupled to a fluid conduit in fluid communication with the generator 110 and be configured to generate a predetermined fluid flow rate through the generator 110 to facilitate formation of a set of micro-organospheres. In some variations, a pump 120 may comprise a positive displacement pump (e.g., a peristaltic pump), a centrifugal pump, or combinations thereof, and the like. In some variations, one or more sample sources 130 may be coupled to the fluid pump 120.

In some variations, the pumps 120 may comprise one or more valves. The pumps 120 may be controlled by the computing device 160 in a predetermined manner. For example, one or more pumps and switches may be serially activated in a predetermined order to ensure consistent processing of samples with repeatable output metrics. In some variations, the pump 120 may be configured to produce a pressure of about 100 mbar to about 1000 mbar.

Platform

In some variations, the micro-organosphere generator 110 may comprise one or more platforms 122 (e.g., moveable stage, tray) configured to position one or more components of the generator 110 relative to each other. For example, the platform 122 may be configured to hold (e.g., secure) the microfluidic device 112 in place relative to the platform 122. The platform 122 may further be configured to move (e.g., with one or more degrees of freedom, translate along a predetermined X-axis and/or Y-axis) so as to position the microfluidic device 112 at a predetermined location relative to the sample source 130, fluid source 134, and imaging device 132. Once in position, the microfluidic device 112 may, for example, receive a light beam from the imaging device 132 that may allow imaging and subsequent data processing. In some variations, the platform 122 may be configured to move a microfluidic device 112 for connection (in fluid communication) with one or more fluid lines coupled to one or more of the sample source 130, fluid source 134, waste vessel 136, or the like. Additionally or alternatively, the platform 122 may be configured to move the fluid lines towards a stationary microfluidic device 114.

Sample Source

In some variations, a sample source 130 may comprise one or more cancer cells, stromal cells, cell lines, non-cancer cells, organoids, patient-derived xenograft, cell mixtures, at controlled or uncontrolled stoichiometry, single cell suspensions, frozen tissue (e.g., biobank), fresh resection, biopsies (e.g., fine needle aspirates), and extracellular matrix (ECM) (e.g., Matrigel®), combinations thereof, and the like. For example, the sample may be derived from a patient such as extracted from a small patient biopsy, (e.g., for quick diagnostics to guide therapy), from resected patient tissue, including resected primary tumor or part of a dysfunctional organ (e.g., for high-throughput screening). The sample tissue (e.g., biopsy) used to form the micro-organospheres (e.g., the dissociated tissue) may be derived from a normal or healthy biological tissue, or from a biological tissue afflicted with a disease or illness, such as a tissue or fluid derived from a tumor. The tissue used in the micro-organospheres may include cells of the immune system, such as T lymphocytes, B lymphocytes, polymorphonuclear leukocytes, macrophages and dendritic cells. In some variations, the cells may be stem cells, progenitor cells or somatic cells. In some variations, the tissue may be mammalian cells such as human cells or cells from animals such as mice, rats, rabbits, combinations thereof, and the like. In some variations, the sample source 130 may comprise preadipocytes, mesenchymal stem cells (MSCs), mast cells, and adipose tissue macrophages, blood vessels and/or microvascular fragments found within a stromal vascular fraction.

In some variations, micro-organospheres may comprise one or more cell types including, but not limited to neurons, cardiomyocytes, myocytes, chondrocytes, pancreatic acinar cells, islets of Langerhans, osteocytes, hepatocytes, Kupffer cells, fibroblasts, myoblasts, satellite cells, endothelial cells, adipocytes, preadipocytes, biliary epithelial cells, combinations thereof, and the like. Cells may be biopsied from one or more of bone marrow, skin, cartilage, tendon, bone, muscle (including cardiac muscle), blood vessels, corneal, neural, brain, gastrointestinal, renal, liver, pancreatic (including islet cells), lung, pituitary, thyroid, adrenal, lymphatic, salivary, ovarian, testicular, cervical, bladder, endometrial, prostate, vulval, or esophageal tissue.

In general, these tissues (and resulting cells) may generally be taken from a biopsy to form the micro-organospheres. Thus, the tissue may be derived from any of a biopsy, a surgical specimen, an aspiration, a drainage, or a cell-containing fluid. Suitable cell-containing fluids include any of blood, lymph, sebaceous fluid, urine, cerebrospinal fluid, or peritoneal fluid. For example, in patients with transcoelomic metastasis, ovarian or colon cancer cells may be isolated from peritoneal fluid. Similarly, in patients with cervical cancer, cervical cancer cells may be taken from the cervix, for example by large excision of the transformation zone or by cone biopsy. In some variations, micro-organospheres may contain multiple cell types that are resident in the tissue or fluid of origin. In some variations, the cells may be obtained directly from the patient without intermediate steps of subculture, or they may first undergo an intermediate culturing step to produce a primary culture.

In some variations, different sample types (e.g., cells, ECM) may be disposed in separate chambers (e.g., tubes, reservoirs, compartments) of the sample source 130 prior to mixing in the microfluidic devices 112. In some variations, the sample sources 130 may be set at a predetermined temperature (e.g., 4° C., between about 4° C. and about 8° C.).

The sample (e.g., a tumor sample) can be dissociated to cells and/or cell clusters before the cells and/or cell clusters are used to form micro-organospheres.

Imaging Device

In some variations, the system 100 may comprise one or more imaging devices 132 configured to generate imaging data processed by a controller (e.g., processor and memory). For example, an imaging device (e.g., camera) may be configured to image one or more of the microfluidic devices 112, polymerizer 140, or demulsifier 150 for monitoring a micro-organosphere forming process. In some variations, one or more characteristics of the mixture and/or micro-organospheres may be estimated based at least on the imaging data. In some variations, temperature and/or pressure of the system may be controlled based at least on the imaging data. For example, the pressure within the micro-fluidic device 112 may be modified in real-time based on the size and shape of micro-organospheres formed that are estimated from the imaging data.

In some variations, the imaging device 132 may comprise a camera, lens, optical sensor (e.g., a charged coupled device (CCD) or complementary metal-oxide semiconductor (CMOS) optical sensor), light source, combinations thereof, and the like. For example, the optical sensor may be a CMOS or CCD array with or without a color filter array and associated processing circuitry. In some variations, a light source (e.g., laser, LED, lamp, or the like) may be configured to generate light that may be carried by fiber optic cables or the imaging device 132 may comprise one or more LEDs configured to provide illumination. For example, the imaging device 132 may comprise a bundle of flexible optical fibers (e.g., a fiberscope). The fiberscope may be configured to receive and propagate light from an external light source.

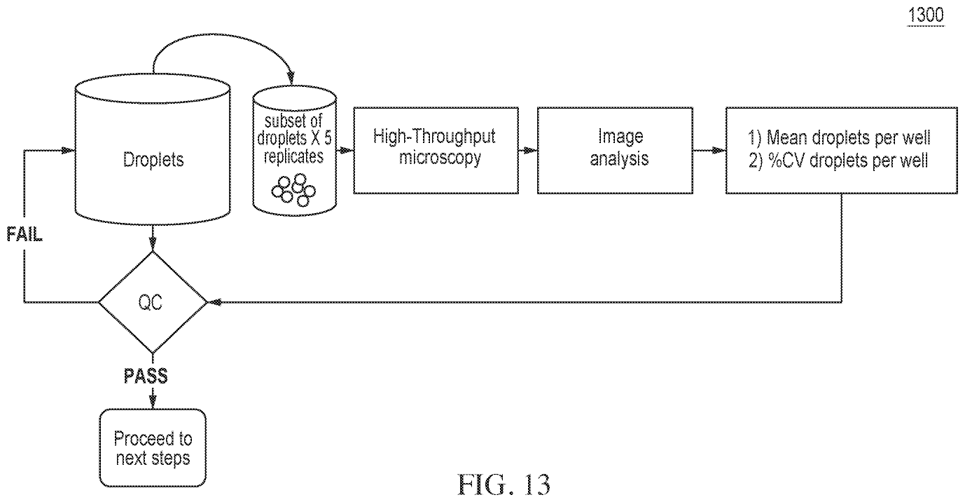

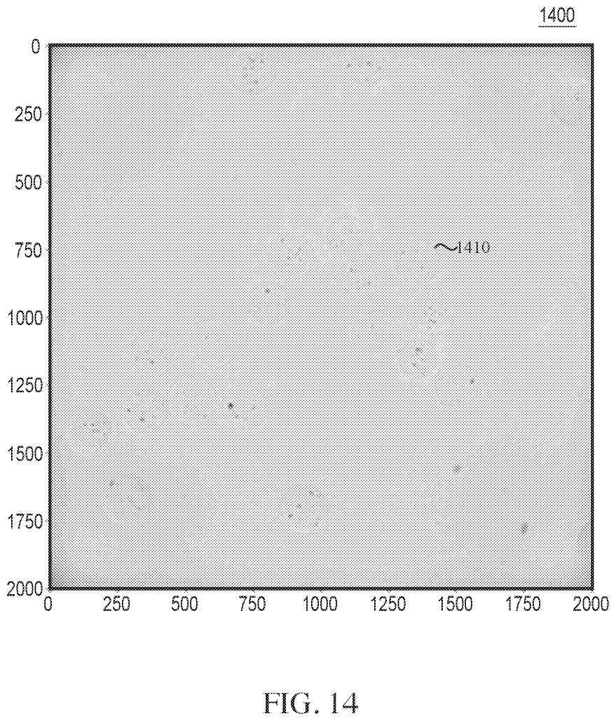

In some variations, the imaging device 132 may comprise one or more microscopy techniques such as confocal microscopy, thus enabling full stack imaging due to the relatively smaller diameter of micro-organospheres compared to organoids. Some conventional biological imaging systems are limited to an imaging depth of about 300 μm. However, organoids typically comprise a depth of between about 1 mm to about 2 mm. Therefore, conventional imaging systems have visual access to about a third or less of an organoid. In some variations, the micro-organospheres may comprise a depth of about 300 μm or less that allow high-throughput imaging of an entire micro-organosphere. Furthermore, the consistency of the micro-organospheres described herein enables their alignment on a single focal plane such that microscopes (e.g., confocal microscopes) may be configured to image a plurality of micro-organospheres simultaneously. In addition, the smaller relative size (e.g., diameter) of micro-organospheres allows for increased spatial density and a higher number of micro-organospheres to be imaged in a single field of view. For example, micro-organospheres may be spherical and have a volume of about 14 nL, which is significantly smaller than the organoids having a half-spherical dome shape and a volume of about 50 μL. As a result, the depth of a micro-organosphere (i.e., along the focal z-axis) may be significantly smaller than organoids. Therefore, micro-organospheres may be imaged using full stack imaging, whereas the thickness of conventional organoids requires image acquisition in multiple planes (e.g., z-stacking) for accurate imaging. Thus, the speed and throughput of conventional micro-organosphere imaging may be improved relative to organoid imaging. FIG. 14 is an image 1400 of a set of identified micro-organospheres 1410 using the imaging devices described herein. FIG. 14 shows an evenly distributed set of cells within each droplet.

Fluid Source

In some variations, the micro-organosphere system 100 may comprise one or more fluid sources 134 including, but not limited to, an extracellular matrix protein (e.g., fibronectin), a drug (e.g., small molecules), a peptide, an antibody (e.g., to modulate any of cell survival, proliferation or differentiation), an inhibitor of a particular cellular function, a reagent, immiscible material (e.g., hydrophobic, oil), a natural gel, a synthetic gel (e.g., hydrogel), a fluid matrix material, combinations thereof, and the like.

In some variations, the fluid matrix material may be configured to form a support or support network for dissociated cells dispersed within it. In some variations, the fluid matrix material may comprise one or more polymers and hydrogels comprising collagen, fibrin, chitosan, Matrigel®, polyethylene glycol, dextrans including chemically crosslinkable or photo-crosslinkable dextrans, and the like, as well as electrospun biological, synthetic, or biological-synthetic blends. For example, the matrix material may be a gel that comprises collagen type 1 such as collagen type 1 obtained from rat tails. In some variations, the gel may be a pure collagen type 1 gel or may be one that contains collagen type 1 in addition to other components, such as other extracellular matrix proteins. A synthetic gel may refer to a gel that does not naturally occur in nature. Examples of synthetic gels include gels derived from any of polyethylene glycol (PEG), polyhydroxyethyl methacrylate (PHEMA), polyvinyl alcohol (PVA), poly ethylene oxide (PEO), and the like.

In some variations, hydrogels may comprise polymeric materials including, but not limited to alginate, collagen (including collagen types I and VI), elastin, keratin, fibronectin, proteoglycans, glycoproteins, polylactide, polyethylene glycol, polycaprolactone, polycolide, polydioxanone, polyacrylates, polyurethanes, polysulfones, peptide sequences, proteins and derivatives, oligopeptides, gelatin, elastin, fibrin, laminin, polymethacrylates, polyacetates, polyesters, polyamides, polycarbonates, polyanhydrides, polyamino acids carbohydrates, polysaccharides and modified polysaccharides, and derivatives and copolymers thereof as well as inorganic materials such as glass such as bioactive glass, ceramic, silica, alumina, calcite, hydroxyapatite, calcium phosphate, bone, combinations thereof, and the like.

In some variations, the fluid source 134 may comprise one or more temperature-controlled compartments. In some variations, the number and quantity of fluids may be sufficient to supply a plurality of manufacturing runs. For example, loading cells may ensure that the level of a fluid source is enough for each run performed.

Waste Vessel

In some variations, the micro-organosphere system 100 may comprise one or more waste vessels 136 fluidically coupled to the micro-organosphere generator 110 and/or the demulsifier 150. The waste vessel 136 may be configured to store waste products from the micro-organosphere formation process such as oil and/or other waste products from the demulsifier 150.

Polymerizer

In some variations, the micro-organosphere system 100 may comprise one or more polymerizers 140 coupled to an output of the microfluidic device 112 and configured to polymerize a mixture (e.g., droplets) to form a set of micro-organospheres (e.g., droplet micro-organospheres). Polymerizing the mixture may increase stability prior to demulsification. In some variations, the polymerizer 140 may be configured to heat the mixture to a predetermined temperature (e.g., about 37° C., between about 10° C. and about 40° C.) for a predetermined amount of time. In some variations, the polymerizer 140 may be integrated with or distinct from the microfluidic device 112. For example, the microfluidic device 112 may be configured to form a mixture at a temperature of about 4° C. The mixture may flow into the polymerizer 140 (e.g., heating chamber) within the microfluidic device 112. The polymerizer 140 may be configured to polymerize the mixture at about 37° C. using a heater of a temperature regulator 118. For example, the temperature regulator 118 may be coupled to a heat conductive material surrounding the polymerizer 140 to evenly distribute heat to the mixture. The polymerizer 140 may comprise one or more temperature sensors configured to generate sensor data for closed loop control of the micro-organosphere formation process. Additionally or alternatively, the polymerizer 140 may comprise chemical polymerization (e.g., using calcium to polymerize the mixture).

Demulsifier

In some variations, the micro-organosphere system 100 may comprise one or more demulsifiers 150. In some variations, the micro-organospheres formed after polymerization may be immersed in a fluid such as oil that may be removed by the demulsifier 150. After separating from oil, growth media may be introduced to the micro-organospheres. In some variations, the demulsifier 150 may comprise a separate microfluidic device configured to filter polymerized droplet micro-organospheres from oil into growth (e.g., cell culture) media.

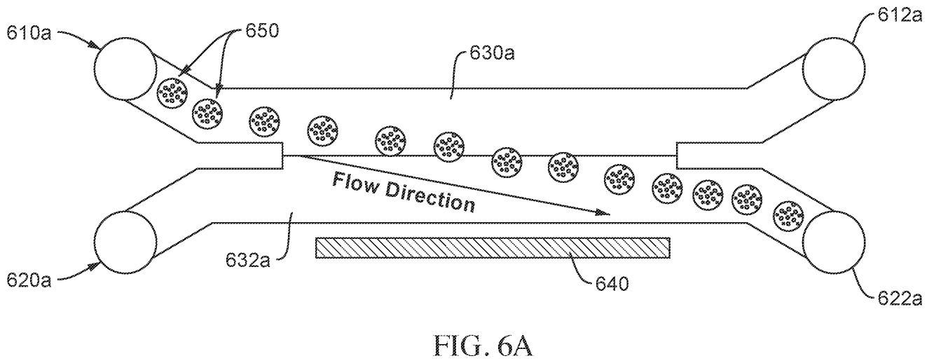

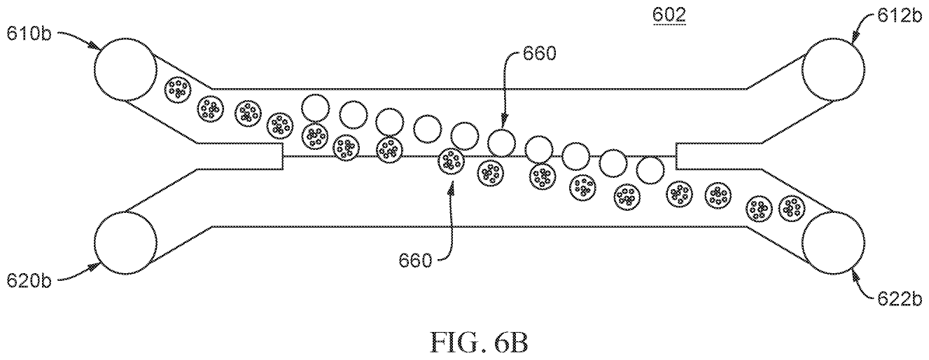

FIG. 6A is a schematic diagram of a demulsifier 600 based on magnetic separation. The demulsifier 600 may comprise a first inlet 610A (e.g., oil and micro-organosphere inlet), first outlet 612A (e.g., oil and waste outlet), second inlet 620A (e.g., growth media and wash inlet), and second outlet 622A (e.g., growth media and micro-organosphere outlet). The first inlet 610A and the second inlet 620A may be disposed on a first side of the demulsifier 600 and the first outlet 612A and the second outlet 622A may be disposed on a second side of the demulsifier 600 opposite the first side. In some variations, a mixture of a first fluid (e.g., oil) and polymerized micro-organospheres 650 may be received in the first inlet 610A. A second fluid (e.g., growth media, wash fluid, aqueous solution) may be received in the second inlet 620A. The demulsifier 600 may be configured for laminar flow, as shown in FIG. 6A, such that the hydrophobic properties of the aqueous fluid from the second inlet 620A and oil from the first inlet 610A do not mix within the demulsifier 600. Instead, a first flow stream 630A (e.g., oil flow stream) and a second flow stream 632A (e.g., aqueous flow stream) are configured to flow through the demulsifier 600 in parallel. In some variations, the demulsifier 600 may comprise a magnet 640 that serves as a flow separator configured to separate the micro-organospheres 650 that contain magnetic nanoparticles from the first flow stream 630A (e.g., oil flow stream). In some variations, the magnet 640 may be configured to extend along a predetermined length of the demulsifier 600. As the micro-organospheres 650 flow through the demulsifier 600, the magnet 640 may be configured to separate the micro-organospheres 650 from the first flow stream 630A (e.g., an oil flow stream) and the second flow stream 632A (e.g., an aqueous flow stream).

FIG. 6B is a demulsifier 602 configured to take advantage of laminar flow properties and small microstructures (e.g., micro-pillars) to filter polymerized droplets from oil into media. The demulsifier 602 may comprise a first inlet 610B (e.g., oil and micro-organosphere inlet), a first outlet 612B (e.g., oil and waste outlet), a second inlet 620B (e.g., growth media and wash inlet), and a second outlet 622B (e.g., growth media and micro-organosphere outlet). The first inlet 610B and the second inlet 620B may be disposed on a first side of the demulsifier 602 and the first outlet 612B and the second outlet 622B may be disposed on a second side of the demulsifier 602 opposite the first side. In some variations, a mixture of a first fluid (e.g., oil) and polymerized micro-organospheres 650 may be received in the first inlet 610B. A second fluid (e.g., growth media, wash fluid, aqueous solution) may be received in the second inlet 620B. In some variations, the demulsifier 602 may be configured for laminar flow, as shown in FIG. 6B, such that the hydrophobic properties of the aqueous fluid from the second inlet 620B and oil from the first inlet 610B do not mix within the demulsifier 602. Instead, a first flow stream (e.g., an oil flow stream (not shown)) and a second flow stream (e.g., an aqueous flow stream (not shown)) flow through the demulsifier 602 in parallel. In some variations, the demulsifier 602 may comprise a flow separator 660 (e.g., a set of micro-pillars, shown as gray filled circles in FIG. 6B) configured to separate micro-organospheres 650 (shown as unfilled circles in FIG. 6B) from the first flow stream (e.g., oil flow stream). The flow separator 660 may be configured to extend along a predetermined length of the demulsifier 602. As the micro-organospheres 650 flow through the demulsifier 602, the micro-pillars of the flow separator 640 may be configured to separate the micro-organospheres 650 from the first flow stream (e.g., oil flow stream) and the second flow stream (e.g., aqueous flow stream). In some variations, the micro-pillars can be positioned at an angle of about one degree from the first flow stream (e.g., oil flow stream) into the second flow stream (e.g., aqueous flow stream), thereby forcing the micro-organospheres 650 from the first flow stream into the second flow stream while allowing each flow stream to remain flowing in parallel. The spacing of the micro-pillars may be such that the micro-organospheres 650 are unable to pass through the micro-pillars and the spacing can be varied in fabrication depending on the expected droplet size.

At a distal end of the demulsifier 600 or 602, the first flow stream may be configured to flow through first outlet 612A or 612B and the second flow stream flow may be configured to flow through the second outlet 622A or 622B. In some variations, the first outlet 612A or 612B may be in fluid communication with a waste vessel (not shown), and the second outlet 622A or 622B may be in fluid communication with an output (e.g., collection vessel) to facilitate recovery of a high percentage of formed micro-organospheres. In contrast to conventional demulsification methods, the demulsifier 600 or 602 as described herein may be configured to demulsify the micro-organospheres automatically without manual handling or centrifugation.

In some variations, demulsification may be based on continuous supernatant assaying. In some variations, individual micro-organospheres within a well (e.g., 96 well plate) may be cultured such that a supernatant may be fractioned off at predetermined intervals. The collected supernatant may be assayed separately.

Output

In some variations, the micro-organosphere system 100 may comprise one or more outputs 152 (e.g., vessels, containers, collectors, wells, assays, or recovery vessel) configured to receive the formed micro-organospheres. In some variations, a predetermined number of micro-organospheres may be dispensed into a plurality of wells. In some variations, the output 152 may be configured to couple to the micro-organospheres. For example, micro-organospheres may be strongly attached to predetermined portions of the well (e.g., predetermined locations at the bottom of a well), thereby enabling high-throughput processing such as rapid media exchanges and fixed imaging with increased resistance to one or more chemical and mechanical treatments.

In some variations, the output 152 (e.g., well plates) may comprise one or more coatings and textures (e.g., patterns). In some variations, the bottom surface of a well may coated and/or patterned to facilitate attachment of the micro-organospheres to the bottom surface. For example, non-specific antibodies may be attached to the bottom of a plate where the antibodies comprise an affinity for proteins or other molecules forming a scaffolding of the micro-organospheres. Consequently, micro-organospheres that contact the said antibodies may be strongly bound to the bottom of the plate. In some variations, one or more plastics may be disposed on a surface of an output (e.g., bottom of a well) and configured to attach (e.g., bond) to micro-organospheres.

Computing Device

In some variations, a system 100 may comprise a computing device 160 comprising a controller (e.g., a processor 162, memory 164), communication device 166, input device 168, display 170, or a combination thereof. The computing device 160 may be configured to control (e.g., operate) the system 100. The computing device 160 may comprise a plurality of devices. For example, the micro-organosphere generator 110 may enclose one or more components of the computing device 160 (e.g., processor 162, memory 164, communication device 166) while one or more components of the computing device 160 may be provided remotely to the micro-organosphere generator 110 (e.g., input device 168 or display 170).

In some variations, the controller may be configured to receive imaging data corresponding to one or more of the mixture or the set of micro-organospheres, and estimate one or more characteristics of the set of micro-organospheres based at least on the imaging data. In some variations, a controller may be configured to receive imaging data corresponding to the set of micro-organospheres, and identify the set of micro-organospheres comprising a diameter of between about 50 μm and about 500 μm based at least on the imaging data.

In some variations, the controller may be configured to estimate a number of micro-organospheres in a plurality of wells based at least on the imaging data.

In some variations, the controller may be configured to receive the sensor data from the one or more sensors, and estimate one or more characteristics of the set of micro-organospheres based at least on the sensor data. In some variations, the controller may be configured to modify one or more of the pump or the temperature based at least on the imaging data and the sensor data.

In some variations, the controller may be configured to estimate one or more characteristics of the mixture based at least on the imaging data. For example, one or more of the characteristics of the mixture comprises a total number of cells and a number of living cells. In some variations, the controller may be configured to estimate one or more characteristics of the biological sample based at least on the imaging data. For example, one or more of the characteristics of the biological sample comprises a total number of cells and a number of living cells.

In some variations the controller may be configured to receive imaging data corresponding to one or more cells, or characteristics of one or more cells, in one or more micro-organosphere.

Processor

The processor (e.g., processor 162) described here may process data and/or other signals to control one or more components of the system (e.g., micro-organosphere generator 110, imaging device 132, or computing device 160). The processor may be configured to receive, process, compile, compute, store, access, read, write, and/or transmit data and/or other signals. Additionally, or alternatively, the processor may be configured to control one or more components of a device and/or one or more components of computing device (e.g., console, touchscreen, personal computer, laptop, tablet, server).

In some variations, the processor may be configured to access or receive data and/or other signals from one or more of micro-organosphere generator 110, imaging device 132, server, computing device 160, or a storage medium (e.g., memory, flash drive, memory card, database). In some variations, the processor may be any suitable processing device configured to run and/or execute a set of instructions or code and may include one or more data processors, image processors, graphics processing units (GPU), physics processing units, digital signal processors (DSP), analog signal processors, mixed-signal processors, machine learning processors, deep learning processors, finite state machines (FSM), compression processors (e.g., data compression to reduce data rate and/or memory requirements), encryption processors (e.g., for secure wireless data transfer), and/or central processing units (CPU). The processor may be, for example, a general purpose processor, Field Programmable Gate Array (FPGA), an Application Specific Integrated Circuit (ASIC), a processor board, and/or the like. The processor may be configured to run and/or execute application processes and/or other modules, processes and/or functions associated with the system. The underlying device technologies may be provided in a variety of component types (e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, and the like.

The systems, devices, and/or methods described herein may be performed by software (executed on hardware), hardware, or a combination thereof. Hardware modules may include, for example, a general-purpose processor (or microprocessor or microcontroller), a field programmable gate array (FPGA), and/or an application specific integrated circuit (ASIC). Software modules (executed on hardware) may be expressed in a variety of software languages (e.g., computer code), including structured text, typescript, C, C++, C #, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

Memory

The micro-organosphere systems and devices described here may include a memory (e.g., memory 164) configured to store data and/or information. In some variations, the memory may include one or more of a random access memory (RAM), static RAM (SRAM), dynamic RAM (DRAM), a memory buffer, an erasable programmable read-only memory (EPROM), an electrically erasable read-only memory (EEPROM), a read-only memory (ROM), flash memory, volatile memory, non-volatile memory, combinations thereof, or the like. In some variations, the memory may store instructions to cause the processor to execute modules, processes, and/or functions associated with the device, such as image processing, image display, sensor data, data and/or signal transmission, data and/or signal reception, and/or communication. Some variations described herein may relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The computer code (also may be referred to as code or algorithm) may be those designed and constructed for the specific purpose or purposes. In some variations, the memory may be configured to store any received data and/or data generated by the computing device and/or imaging device. In some variations, the memory may be configured to store data temporarily or permanently.

Input Device

In some variations, the display may include and/or be operatively coupled to an input device 168 (e.g., touch screen) configured to receive input data from a user. For example, user input to an input device 168 (e.g., keyboard, buttons, touch screen) may be received and processed by a processor (e.g., processor 162) and memory (e.g., memory 164) of the system 100. The input device may include at least one switch configured to generate a user input. For example, an input device may include a touch surface for a user to provide input (e.g., finger contact to the touch surface) corresponding to a user input. An input device including a touch surface may be configured to detect contact and movement on the touch surface using any of a plurality of touch sensitivity technologies including capacitive, resistive, infrared, optical imaging, dispersive signal, acoustic pulse recognition, and surface acoustic wave technologies. In variations of an input device including at least one switch, a switch may have, for example, at least one of a button (e.g., hard key, soft key), touch surface, keyboard, analog stick (e.g., joystick), directional pad, mouse, trackball, jog dial, step switch, rocker switch, pointer device (e.g., stylus), motion sensor, image sensor, and microphone. A motion sensor may receive user movement data from an optical sensor and classify a user gesture as a user input. A microphone may receive audio data and recognize a user voice as a user input.

In some variations, the micro-organosphere system may optionally include one or more output devices in addition to the display, such as, for example, an audio device and haptic device. An audio device may audibly output any system data, alarms, and/or notifications. For example, the audio device may output an audible alarm when a malfunction is detected. In some variations, an audio device may include at least one of a speaker, piezoelectric audio device, magnetostrictive speaker, and/or digital speaker. In some variations, a user may communicate with other users using the audio device and a communication channel. For example, a user may form an audio communication channel (e.g., VoIP call).

Additionally or alternatively, the system may include a haptic device configured to provide additional sensory output (e.g., force feedback) to the user. For example, a haptic device may generate a tactile response (e.g., vibration) to confirm user input to an input device (e.g., touch surface). As another example, haptic feedback may notify that user input is overridden by the processor.

Communication Device

In some variations, the computing device may include a communication device (e.g., communication device 166) configured to communicate with another computing device and one or more databases. The communication device may be configured to connect the computing device to another system (e.g., Internet, remote server, database) by wired or wireless connection. In some variations, the system may be in communication with other devices via one or more wired and/or wireless networks. In some variations, the communication device may include a radiofrequency receiver, transmitter, and/or optical (e.g., infrared) receiver and transmitter configured to communicate with one or more devices and/or networks. The communication device may communicate by wires and/or wirelessly.

The communication device may include RF circuitry configured to receive and send RF signals. The RF circuitry may convert electrical signals to/from electromagnetic signals and communicate with communications networks and other communications devices via the electromagnetic signals. The RF circuitry may include well-known circuitry for performing these functions, including but not limited to an antenna system, an RF transceiver, one or more amplifiers, a tuner, one or more oscillators, a digital signal processor, a CODEC chipset, a subscriber identity module (SIM) card, memory, and so forth.

Wireless communication through any of the devices may use any of plurality of communication standards, protocols and technologies, including but not limited to, Global System for Mobile Communications (GSM), Enhanced Data GSM Environment (EDGE), high-speed downlink packet access (HSDPA), high-speed uplink packet access (HSDPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (WiFi) (e.g., IEEE 802.11a, IEEE 802.11b, IEEE 802.11g, IEEE 802.11n, and the like), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), EtherCAT, OPC Unified Architecture, or any other suitable communication protocol. In some variations, the devices herein may directly communicate with each other without transmitting data through a network (e.g., through NFC, Bluetooth, WiFi, RFID, and the like).

In some variations, the systems, devices, and methods described herein may be in communication with other wireless devices via, for example, one or more networks, each of which may be any type of network (e.g., wired network, wireless network). The communication may or may not be encrypted. A wireless network may refer to any type of digital network that is not connected by cables of any kind. Examples of wireless communication in a wireless network include, but are not limited to cellular, radio, satellite, and microwave communication. However, a wireless network may connect to a wired network in order to interface with the Internet, other carrier voice and data networks, business networks, and personal networks. A wired network is typically carried over copper twisted pair, coaxial cable and/or fiber optic cables. There are many different types of wired networks including wide area networks (WAN), metropolitan area networks (MAN), local area networks (LAN), Internet area networks (IAN), campus area networks (CAN), global area networks (GAN), like the Internet, and virtual private networks (VPN). Hereinafter, network refers to any combination of wireless, wired, public and private data networks that are typically interconnected through the Internet, to provide a unified networking and information access system.

Cellular communication may encompass technologies such as GSM, PCS, CDMA or GPRS, W-CDMA, EDGE or CDMA2000, LTE, WiMAX, and 5G networking standards. Some wireless network deployments combine networks from multiple cellular networks or use a mix of cellular, Wi-Fi, and satellite communication.

Display

Image data may be output on a display (e.g., display 170) of a micro-organosphere system 100. In some variations, a display may include at least one of a light emitting diode (LED), liquid crystal display (LCD), electroluminescent display (ELD), plasma display panel (PDP), thin film transistor (TFT), organic light emitting diodes (OLED), electronic paper/e-ink display, laser display, and/or holographic display. In some variations, the display 170 may be integrated as a touch screen of the micro-organosphere generator 110.

### II. Methods

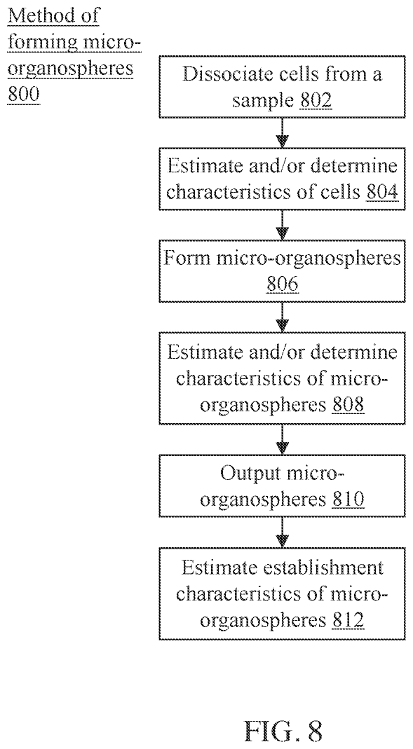

Described here are methods of forming micro-organospheres using the automated micro-organosphere systems and devices described herein. For example, the micro-organospheres may be formed, identified, and estimated in a single end-to-end integrated workflow comprising microfluidic and microfluidic elements. Furthermore, the identified micro-organospheres may be precisely distributed based on one or more micro-organosphere characteristics to enable, for example, rapid drug screening. FIG. 8 is a flowchart that generally describes a variation of a method of forming micro-organospheres. The method 800 may include dissociating cells from a sample (e.g., patient-derived tissue sample) 802. In some variations, the cells may be dissociated using one or more of mechanical digestion, enzymatic digestion, combinations thereof, or the like. Any tissue type may be processed. In some variations, the cells may be mixed to form, for example, a cell and Matrigel® based mixture. In some variations, the cells may also be mixed to form a cell and any alternative to Matrigel described herein.

In some variations, one or more cell characteristics may be estimated and/or determined, according to step 804. For example, a portion of the dissociated cells may be stained (e.g., AO/PI) to estimate a number of living cells and a number of dead cells. These cell estimates allow for micro-organospheres to be formed at a predetermined concentration (e.g., number of living cells per unit volume). Estimating cell characteristics such as seeding density from the sample may enable samples having a number of dead cells (or density) above a predetermined threshold to be rejected. In some variations, micro-organospheres having a predetermined number of living cells may be formed based on the concentration and by controlling the size (e.g., diameter) of the micro-organosphere formed. The number of living cells may be used to determine a volume of fluid matrix material to form a predetermined concentration of mixture.

In some variations, a set of micro-organospheres may be formed, according to step 806. In some variations, the cells may undergo rapid encapsulation to form droplets having a predetermined spatial distribution of the mixture. In some variations, a size (e.g., diameter) of the droplets may be controlled (e.g., based on temperature and pressure) to form micro-organospheres comprising a predetermined number of cells and concentration.

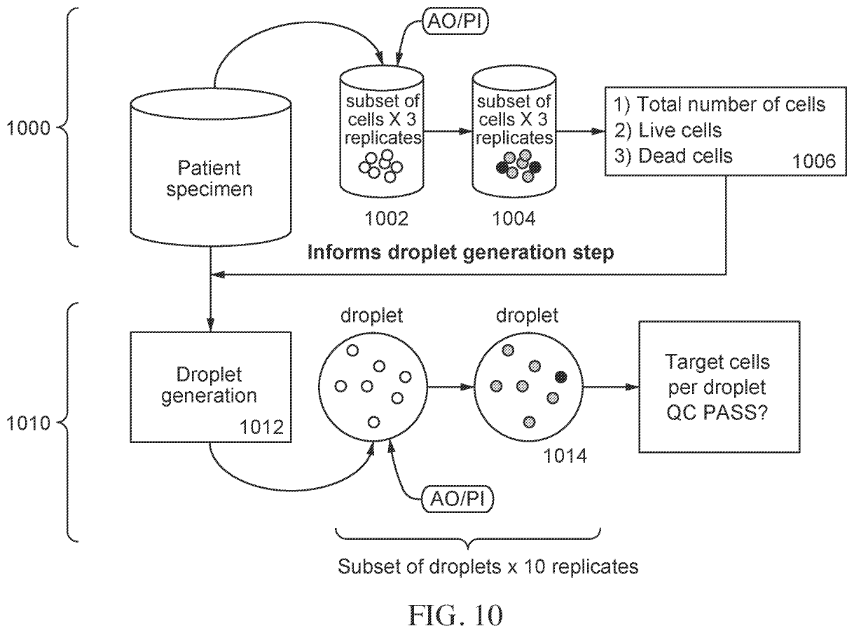

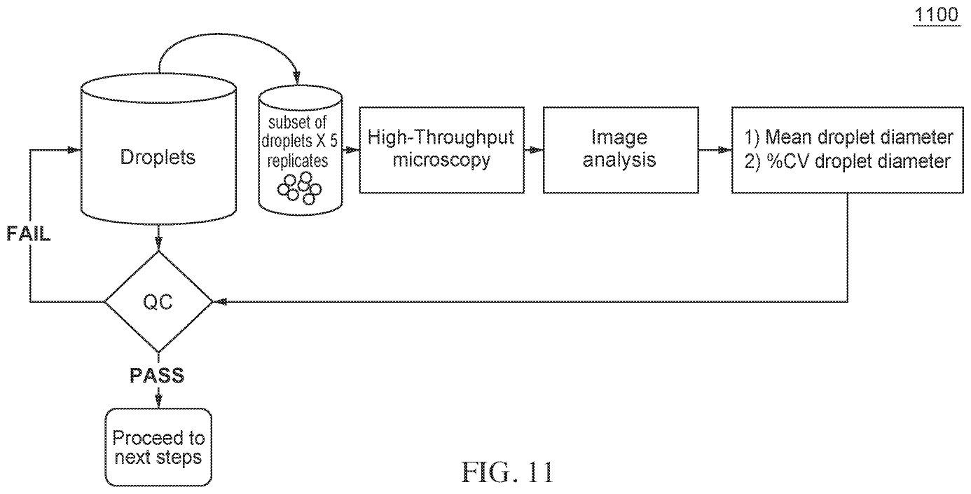

In some variations, one or more micro-organosphere characteristics may be estimated and/or determined, according to step 808. In some variations, a number of micro-organospheres per unit volume may be estimated. The estimate may be used to ensure that the cell type composition and the expected number of viable cells meets predetermined criteria, and allows the number of cells per droplet post-encapsulation to be controlled. For example, a portion of the formed micro-organospheres may be stained (e.g., AO/PI staining) to determine the number of living and dead cells. FIGS. 10 and 11, as described in more detail herein, illustrates variations of an estimation process for dissociated cells and droplets. In some variations, micro-organosphere formation may correspond to a Poisson sampling distribution of the number of cells per droplet. In some variations, the set of micro-organospheres may be agitated in solution while imaging to improve the estimation of micro-organosphere characteristics.

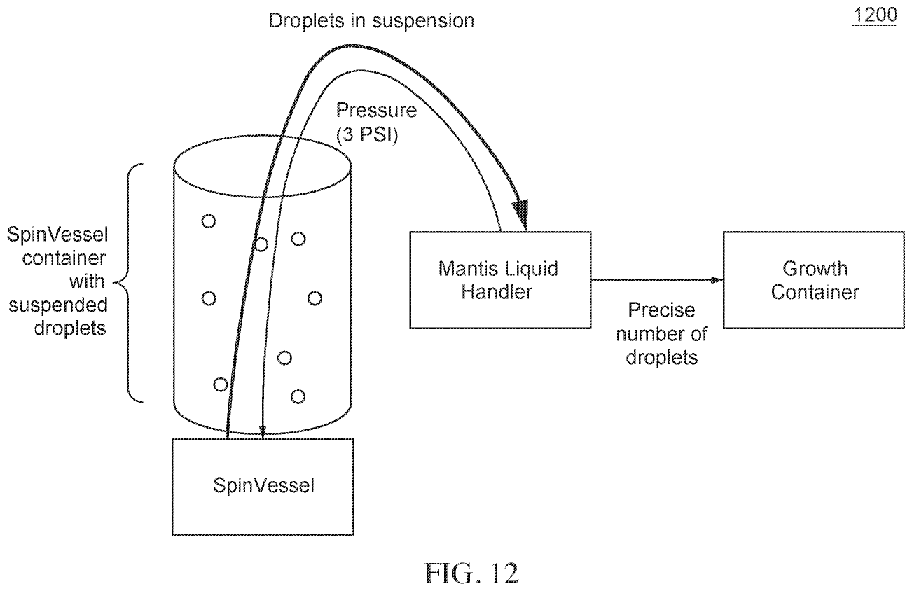

In some variations, the set of micro-organospheres may be output, according to step 810. For example, the set of micro-organospheres may be used for drug assay plating. In some variations, one or more of agitation or controlled dispensing in one or more vessels (e.g., wells, receptacles, containers) may enable a Poisson sample distribution. FIG. 12 illustrates one variation of an agitation and dispensing process, as described in more detail herein. In some variations, imaging data of the wells may be generated to estimate the number and location of the output micro-organospheres as a baseline, for example. In some variations, the number of droplets per well may be used for normalization of quantitative assay results. In some variations, one or more wells may be rejected if the number of droplets within the well does not meet a predetermined range.

In some variations, the micro-organospheres may be agitated to ensure uniform distribution in suspension within growth media while the micro-organospheres are output to, for example, a well plate (e.g., assay well in a cell culture vessel, 6 well plate to 1536 well plate). For example, shaking flasks, manual pipetting, rockers, and the like may be used to ensure even distribution of micro-organospheres. In some variations, the set of micro-organospheres may be output using one or more of pipetting or a liquid handler. For example, a liquid handler may be configured to pipette directly from an agitated vessel comprising the micro-organosphere.

In some variations, one or more establishment characteristics of the set of micro-organospheres may be estimated, according to step 812. For example, imaging at periodic intervals may confirm establishment of the set of micro-organospheres and may enable, for example, the start of a drug assay within about 2 days to about 8 days after seeding the micro-organospheres based on predetermined criteria. By contrast, drug screening using conventional organoids may require about 6 weeks to about 8 weeks to generate and form organoids having a sufficient number of cells for testing a drug response, which may be time consuming and expensive, especially as a diagnostic tool. In some variations, the micro-organospheres described herein may provide drug assay results in less than about 2 weeks. FIG. 13 illustrates one variation of an estimation process for droplets disposed in wells, as described in more detail herein.

In some variations, an imaging device may be configured to generate imaging data of a set of wells (e.g., each well of a well plate). A processor may be configured to estimate the surface area and volume of the cells over time based on the imaging data using one or more computer vision techniques. For example, establishment characteristics of the micro-organospheres may comprise one or more of size, volume, or growth rate of micro-organospheres. In some variations, a micro-organosphere may be identified as established based on one or more predetermined thresholds (e.g., median area of 70 μm², doubling of initial cellular mass).

In some variations, imaging data may be analyzed to identify one or more objects within each micro-organosphere having a diameter greater than a predetermined threshold (e.g., expected diameter of a single cell). Therefore, only multicellular or organoid bodies may be identified. For example, a surface area (e.g., μm 2) of each object identified within each micro-organosphere may be estimated and tracked over time (e.g., hours, days). This approach may generate quantitative data and at high sensitivity to determine establishment and enable drug dosing administration.

In some variations, micro-organospheres may be formed in under about a day and may establish in about 2 days to about 8 days after formation. A drug assay using the micro-organospheres may be run in about 4 days or less, thereby enabling a functional diagnostic in under about 14 days using the systems and methods described herein.

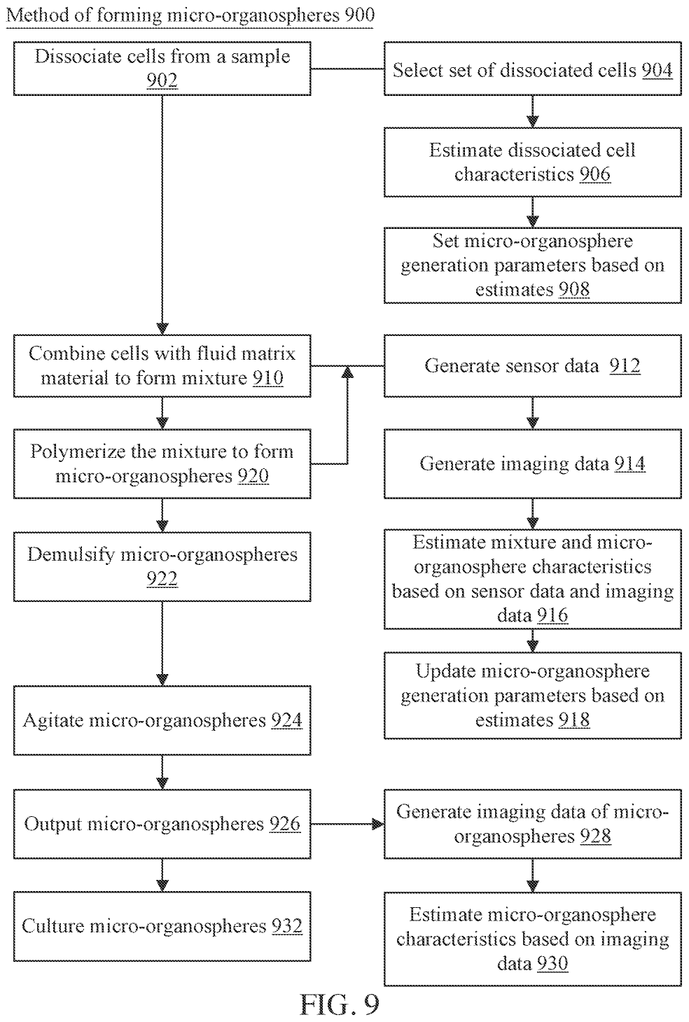

FIG. 9 is a flowchart that generally describes a variation of a method of forming micro-organospheres. In some variations, the method 900 may include dissociating cells from a sample (e.g., patient-derived tissue sample), according to step 902. For example, the sample may be dissociated mechanically and/or chemically (e.g., enzyme treatment). Mechanical dissociation may comprise disrupting connections between associated cells, for example, using a scalpel or scissors or by using a machine such as a homogenizer. Chemical dissociation may comprise treating the cells with one or more enzymes to disrupt connections between associated cells, including for example any of collagenase, dispases, DNAse, and/or hyaluronidase. One or more enzymes may be used under different reaction conditions, such as incubation at 37° C. in a water bath or at room temperature.

In some variations, dissociated tissue may be treated to remove dead and/or dying cells and/or cell debris. The removal of such dead and/or dying cells may include one or more of beads, filtration, antibody methods, combinations thereof, or the like. For example, Annexin V-Biotin binding followed by binding of biotin to streptavidin magnetic beads may enable the separation of apoptotic cells from living cells.

In some variations, the sample from a patient may be from a biopsy (e.g., using a biopsy needle or punch). For example, the biopsy may be taken with a 14-gauge, a 16-gauge, an 18-gauge, etc. needle that may be inserted into the patient tissue to remove the biopsy.

In some variations, dissociated cells may be suspended in a carrier material. In some variations, the carrier material may comprise a fluid matrix material. In some variations, the carrier material may be a material that has a viscosity level configured to delay sedimentation of cells in a cell suspension prior to polymerization and formation of micro-organospheres. In some variations, a carrier material may have sufficient viscosity to allow the dissociated biopsy tissue cells to remain suspended in the suspension until polymerization. In some variations the unpolymerized material may be flowed or agitated in order to keep the cells in suspension and/or distributed as desired.

In some variations, a set of dissociated cells may be selected for analysis, according to step 904. In some variations, one or more characteristics of the selected set of dissociated cells may be estimated, according to step 906. For example, as shown in method 1000 of FIG. 10, the selected set (e.g., subset) of dissociated cells 1002 may be counted and stained 1004 with one or more live/dead stain. Non-limiting examples of live/dead stains include calcein AM (live), ethidium homodimer (dead), trypan blue (live), Hoechst (nuclear), and acridine orange (AO) and propidium iodide (PI) (AO/PI). AO/PI is a fluorescent-based, cell viability assay in which live cells fluoresce green (e.g., 526 nm maximum emission wavelength) and dead cells fluoresce red (e.g., 617 nm maximum emission wavelength). The assay output 1006 may comprise the total number of cells, the total number of viable cells, and the total number of dead cells in the patient cell sample.

In some variations, one or more micro-organosphere generation parameters may be set based on the estimated characteristics of the set of dissociated cells, according to step 908. For example, the results of method 1000 of FIG. 10 may be used to inform the micro-organosphere generation parameters of method 1010, thereby enabling a predetermined number of viable cells 1012 to mix with a predetermined volume of fluid such that a target number of viable cells are disposed within each micro-organosphere 1014. The micro-organosphere generation parameters may comprise one or more of fluid flow rate, temperature, pressure, or the like.

In some variations, the dissociated cells may be combined with fluid matrix material to form a mixture (e.g., unpolymerized mixture), according to step 910. For example, the mixture may comprise the dissociated cells suspended within the mixture. In some variations, the cells may remain suspended and unpolymerized at a predetermined temperature (e.g., between about 1° C. and about 210° C.). The unpolymerized mixture may be dispensed as droplets into an immiscible material, such as an oil. The size and shape of the droplets may correspond to the size and shape of the formed micro-organospheres. For example, uniformly-sized droplets may be formed by combining a stream of the unpolymerized material into one or more (e.g., two converging) streams of the immiscible material (e.g., oil) so that the flow rates and/or pressures of the two streams may determine how droplets of the unpolymerized material are formed as they intersect the immiscible material.

In some variations, the size (e.g., diameter) of the micro-organospheres may be controlled based on one or more of the pressures in the system or viscosities of the materials. Changes in either parameter will alter the consistency in the size (e.g., diameter) of the formed micro-organospheres. For example, time is required for the pressures across the system to stabilize when the various liquids (e.g., cells and fluid matrix material) reach an intersection (e.g., T-junction) and combine. Changes to pressure will create variability in droplet sizes. For example, air bubbles introduced into a microfluidic generator (e.g., microfluidic chap) may change the pressure within the system and thus the size (e.g., diameter) of the micro-organospheres formed.

Additionally or alternatively, one or more droplets may be formed by printing (e.g., by printing droplets onto a surface). For example, the droplets may be printed onto a surface, such as a flat or shaped surface, and polymerized. In some variations, the droplets may be formed using an automatic dispenser (e.g., pipetting device) adapted to release a predetermined amount of the unpolymerized mixture onto a surface, into the air, and/or into a liquid medium (including an immiscible fluid).

Introduction of Non-Cellular Objects into Micro-Organospheres

Additionally or alternatively, step 910 may include forming a mixture by combing one or more non-cellular objections. For example, the micro-organospheres can include one or more non-cellular objects. In some variations, the non-cellular objects can be added to the mixture of cells prior to micro-organosphere formation. In some variations, the non-cellular objects can also be incorporated into the micro-organospheres after they are formed. In some variations, the non-cellular object may comprise an inert particle.

In some variations, each micro-organosphere can include about 1 to about 10,000 non-cellular objects, e.g., about 10 to about 7,500, about 10 to about 5,000, about 100 to about 2,500 non-cellular objects.

In some variations, the non-cellular objects can serve as identifiers (e.g., barcodes) for identifying the micro-organospheres. In the absence of any means for identifying the micro-organospheres, if a particular well is imaged before and after the micro-organospheres have moved, it can be difficult or impossible to match the micro-organospheres in the first and second sets of images for time lapse imaging. The introduction of identifiers into the micro-organospheres can thus overcome this challenge and permit time lapse imaging. In some variations, the non-cellular objects added as identifiers do not affect biological processes.

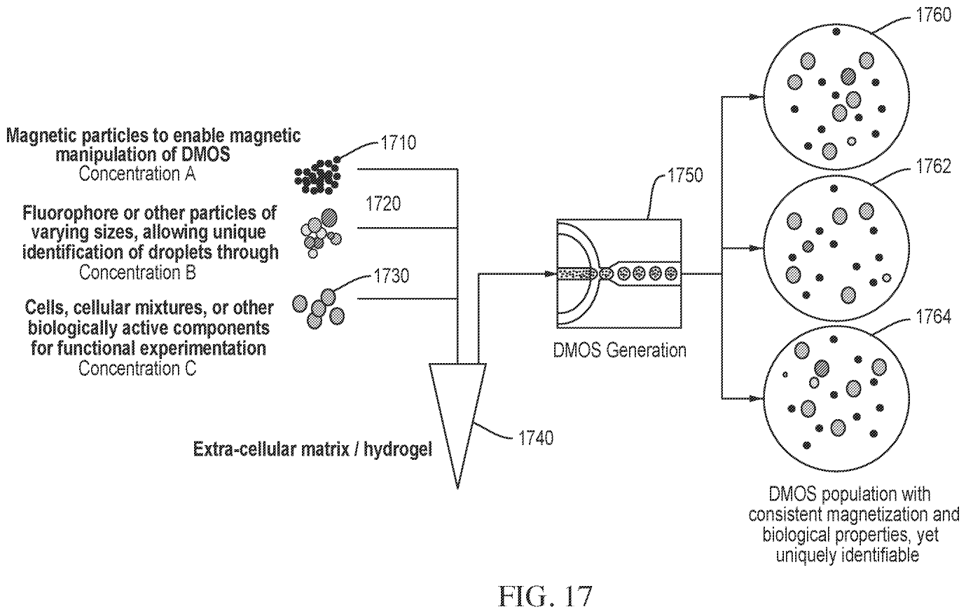

In some variations, the non-cellular objects can comprise particles of difference sizes, photophores, fluorophores, fluorescent particles, colored particles, magnetic particles, and/or magnetizable particles. As shown in FIG. 17, in some variations, to generate unique identifiers (e.g., barcodes), a highly variable source of Type A particles 1710 (e.g., magnetic particles and/or magnetizable particles) and/or Type B particles 1720 (e.g., particles of difference sizes, photophores, fluorophores, fluorescent particles, and/or colored particles) may be introduced into a mixture of Type C particles 1730 (e.g., cells, cellular mixtures, biologically active components) prior to micro-organosphere formation. For example, stochastic sampling of Type A and/or Type B particles during the mixing process may generate a uniquely identifiable combination of Type A and/or Type B particles in each micro-organosphere, effectively serving as a unique identifier of each micro-organosphere. The combination of Type A, B, and C particles may be combined in an extracellular matrix/hydrogel 1740 for micro-organosphere generation 1750 to generate a plurality of sets of micro-organospheres 1760, 1762, 1764. One or more identifiers may be read on a microscopy system and decoded visually or algorithmically, thereby enabling single micro-organosphere tracking across workflows, reformatting steps, mechanical manipulation, and various other applications such as flow cytometry. In some applications, an identifier permits high-throughput sorting of the micro-organospheres.

In some variations, the magnetic particles may comprise one or more ferromagnetic, paramagnetic, or other kind of magnetic particles. In some variations, the magnetic particles may comprise Fe₃O₄. For example, the magnetic particles permit mechanical manipulation and control of micro-organospheres through the use of magnets, thereby allowing increased efficiencies and capabilities at various steps of the workflow, such as phase separation/demulsification, rapid media exchange, and micro-organosphere concentrations at specific locations. For example, the magnetic particles can permit concentrating the micro-organospheres as a single layer at the bottom of a well or culture plate for imaging, at the center of the well or culture plate for imaging of one or more micro-organospheres, and/or at specific locations to facilitate recovery of a set of the micro-organospheres.

Cellular encapsulation may include various forms. In a first variation, a single cell suspension may be added to the extracellular matrix to form micro-organospheres comprising a predetermined number of cells spread throughout the droplet. In a second variation, cells may be encapsulated in multiple steps in order to create a high-density core of cells within a larger extracellular matrix droplet. In these variations, single-cells may be re-suspended in an extracellular matrix at a higher concentration than the first variation and encapsulated in droplets that are significantly smaller than in the first variations. These droplets may be polymerized and re-suspended in a fresh unpolymerized extracellular matrix. This suspension may be processed again form new droplets of about the same size as the first variation that contain a single high-density polymerized cellular core.

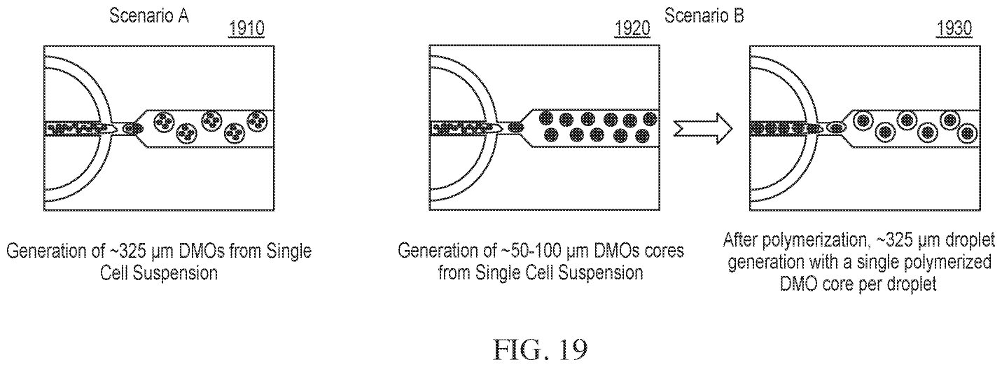

As shown in FIG. 19, micro-organosphere generation can include one or more forms of cellular encapsulation. In Scenario A (1910), a single cell suspension can be added to the extracellular matrix and the micro-organosphere generator may be configured to generate a set of micro-organospheres comprising a predetermined number of cells spread throughout the droplet when generated. These droplets can then be polymerized and analyzed. Alternatively, in Scenario B (1920, 1930), cells can be encapsulated in multiple steps in order to create a high-density core of cells within a larger extracellular matrix droplet. In Scenario B, single cells may be resuspended in an extracellular matrix at a higher concentration than Scenario A and be encapsulated in droplets that are significantly smaller than Scenario A. These droplets can be polymerized and resuspended in a fresh unpolymerized extracellular matrix. This suspension can be processed again on the micro-organosphere generator to create new droplets of the same size as Scenario A that contain a single high-density polymerized cellular core. These droplets can then follow the same manipulation and analysis procedure as Scenario A.

In some variations, the micro-organospheres can be immobilized to a surface. For example, the micro-organospheres can be immobilized to the bottom of a well or culture plate.

If the micro-organospheres include magnetic and/or magnetizable particles, a magnetic force can be used to immobilize the micro-organospheres at a predetermined location. For example, the magnetic force can be applied to the center of the well. Data acquisition may be efficient if using a high-throughput imager if all micro-organospheres can be captured in the field of view and focused on the center of a well.

In some variations, the micro-organospheres can also be immobilized at a specific location through biochemical means, which may comprise exploiting the chemical composition of extracellular matrix gels to capture gel-based micro-organospheres with antibodies that specifically bind the extracellular matrix gels.

In some variations, antibodies can be immobilized to the bottom of a well or culture plate in at least two ways. In some variations, the antibodies may be directly bound to an untreated polystyrene culture dish surface by incubation in a high pH, low ionic strength buffer such as phosphate-buffered saline (PBS). In this environment, antibodies may preferentially bind to the surface through hydrophobic interactions with polystyrene.

In some variations, culture plates may be pre-coated with protein A or protein G before the antibodies are attached. Since protein A/G bind to the Fc region of antibodies, this approach can properly orient the variable regions of antibodies towards the bulk solution and away from the plate surface. In some variations, antibodies can be incubated in a high pH, low ionic strength buffer such as PBS to induce binding to the plates.

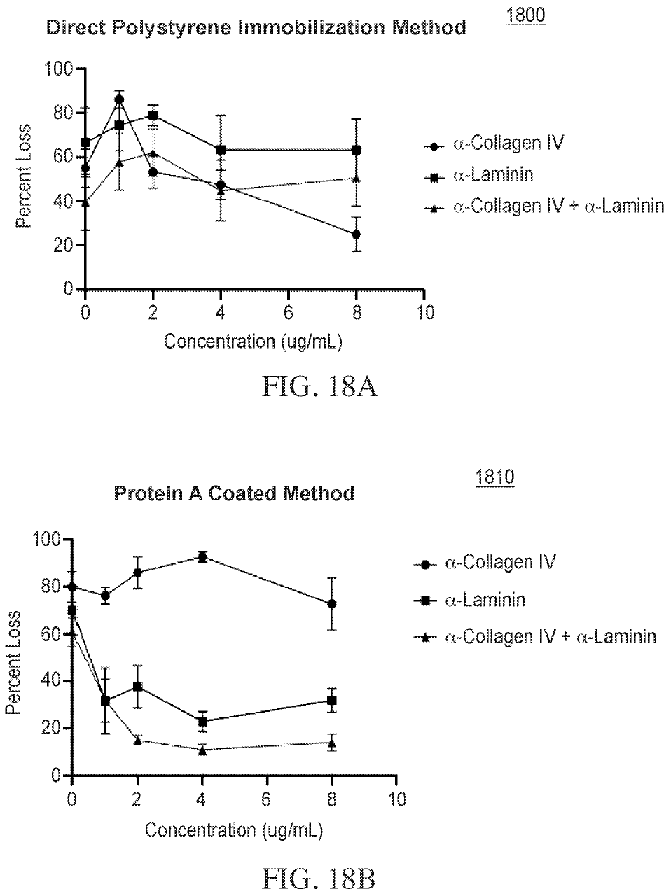

In some variations, approximately 1-10 ug of antibodies can be immobilized to the plate surface after washing. Some extracellular matrix gels are composed of significant proportions of collagen IV and laminin. Culture plates derivatized with anti-mouse laminin and/or anti-mouse collagen IV antibodies, and incubated with Matrigel®-based micro-organospheres may result in their immobilization to the culture plate via the antibodies. For example, FIGS. 18A and 18B are graphs depicting a derivatization of culture plates with mouse anti-laminin and/or mouse anti-collagen IV antibodies via either direct binding to polystyrene (FIG. 18A) or protein A-mediated attachment (FIG. 18B) that results in immobilization of Cultrex-based micro-organospheres in an antibody-concentration-dependent manner, minimizing loss realized due to a media change. Data is plotted as mean with error expressed as SEM of 5 replicate measurements of droplets per well in a 96-well plate. In some variations, following incubation for at least 16 hours at 37 degrees C., a supernatant exchange can be performed with minimal micro-organosphere loss.

In some variations, the micro-organospheres can be deposited at the bottom of the well or culture plate by centrifugation. With specific geometries, the micro-organospheres can be concentrated at predetermined locations.

In some variations, the micro-organospheres can be localized at predetermined locations on the bottom of the well or culture plate. For example, patterns can be made on the bottom of the well or culture plate to facilitate localization. In some variations, dimples can be etched on the bottom of the well or culture plate to increase affinity to the micro-organospheres. In some variations, after coating the bottom of the well or culture plate with a material with affinity to the micro-organospheres, a laser etcher can be used to remove the coating from all the locations where micro-organospheres would be unwanted. The patterns are not limited to the well or culture plate, and can be applied to other vessels.

In some variations, sensor data corresponding to the micro-organosphere forming process (e.g., cells, fluid matrix material, mixture) may be generated, according to step 912. For example, sensor data may be generated by one or more sensors 116 of system 100 described in more detail herein.

In some variations, imaging data corresponding to the micro-organosphere forming process (e.g., cells, fluid matrix material, mixture) may be generated, according to step 914. For example, an imaging device 132 as described herein may be configured to generate imaging data corresponding to mixture formation (e.g., intersection of cells and fluid matrix material at an intersection or junction). In some variations, the formed micro-organospheres may be imaged for further analysis.

In some variations, one or more characteristics of the mixture and/or micro-organosphere may be estimated based on the sensor data and/or imaging data, according to step 916. For example, the imaging data may be processed to generate a size distribution of the droplets formed. In some variations, one or more characteristics may comprise one or more of number of cells per droplets, distribution of cells within the droplet, size (e.g., diameter) of droplet, or the like.

In some variations, micro-organosphere characteristics may be estimated through the acquisition of imaging data without sensor data.

In some variations, micro-organosphere characteristics may be estimated based on the imaging data. For example, step 1010 of FIG. 10 illustrates that a subset of formed micro-organospheres may be stained with AO/PI to count the number of live and dead cells per micro-organosphere. The estimated characteristics may enable runs having, for example, a number of dead cells (or density) above a predetermined threshold to be rejected.

The systems and devices described herein may allow formation of micro-organospheres comprising a predetermined number of cells. For example, a set of 25 runs targeting 20 cells per micro-organospheres formed micro-organospheres having a mean live cells per droplet of 19.3, and live cells per droplet % CV (intra-run and inter-run) of 13.3% and 13.1%, respectively.

With respect to diameter, FIG. 11 illustrates that a subset of formed micro-organospheres (e.g., about 100 droplets) may be imaged using high-throughput microscopy. Image analysis may generate an estimate of mean diameter and variance (% CV). Runs that fall outside a predetermined range of diameters may be rejected.

The systems and devices described herein may allow formation of micro-organospheres comprising a predetermined diameter. For example, a set of 42 runs targeting a 300 μm cell formed micro-organospheres having a mean diameter of 302 μm, and a droplet diameter % CV (intra-run and inter-run) of 20.2% and 11.1%, respectively.

Back to method 900, in some variations, one or more micro-organosphere generation parameters may be updated based on the estimated characteristics, according to step 918. For example, the temperature and/or fluid flow rate of the sample and fluids may be adjusted based on the estimated characteristics. This enables closed-loop control of a micro-organosphere formation process to increase efficiency and yields. In some variations, imaging data without sensor data may be used to estimate the characteristics. One or more of the temperature, fluid flow rate, and/or other conditions may be adjusted based on the estimated characteristics.

In some variations, the mixture may be polymerized to form a set of micro-organospheres, according to step 920. In some variations, the mixture (e.g., droplets) may be polymerized to form the micro-organospheres in an immiscible material (e.g., oil). For example, the immiscible material may be heated to a temperature that causes the unpolymerized mixture (e.g., the fluid matrix material in the unpolymerized material) to polymerize.

In some variations, the set of micro-organospheres may be demulsified, according to step 922. For example, the micro-organospheres may be separated from the immiscible fluid by washing to remove an immiscible fluid and/or by using the demulsifier 600 described with respect to FIG. 6A or demulsifier 602 described with respect to FIG. 6B.

In some variations, the set of micro-organospheres may be agitated, according to step 924. For example, FIG. 12 illustrates a set of micro-organospheres suspended in solution and being agitated to more evenly distribute the micro-organospheres.

In some variations, the set of micro-organospheres may be output, according to step 926. In some variations, the droplets may be dispensed using pressure, sound, charge, combinations thereof, and the like. For example, as shown in FIG. 12, a liquid handler may be used to dispense a predetermined volume of micro-organospheres (at a predetermined concentration) to a growth container (e.g., 96-well plate, 384-well plate, 1536-well plate). Controlling the total cell mass dispensed in a well, for example, may facilitate quantitative measurements relying on cell activity.

In some variations, imaging data of the set of micro-organospheres may be generated, according to step 928. For example, the micro-organospheres in each well of a well plate may be imaged and analyzed. In some variations, one or more characteristics of a set of micro-organospheres may be estimated based on the imaging data, according to step 930. For example, FIG. 13 illustrates that a subset of output micro-organospheres may be imaged using high-throughput microscopy. Image analysis may generate an estimate of a mean number of micro-organospheres per well and variance (% CV). Runs that fall outside a predetermined range of values may be rejected.

In some variations, droplet diameter may be controlled by the configuration of the system, the ratio of sample to matrix (such as Matrigel®) and the number of cells per droplet. Droplet diameter may be monitored by measuring the average droplet size and variance (% CV) after every micro-organosphere formation run via high-throughput microscopy and image analysis. In some variations, a sampling of about 100 droplets may be imaged to estimate the mean droplet size and variance. Runs that do not pass the mean and variance thresholds may be repeated.

The systems and devices described herein may allow formation of micro-organospheres comprising a predetermined diameter. For example, a set of 42 runs targeting a 300 μm cell formed micro-organospheres having a mean diameter of 302 μm, and a droplet diameter % CV (intra-run and inter-run) of 20.2% and 11.1%, respectively.

The systems and devices described herein may allow output of a predetermined number of micro-organospheres per well. For example, a set of runs targeting 30 micro-organospheres per well had a mean number of micro-organospheres per well of 31.2, and a number of droplets per well % CV (intra-run and inter-run) of 20.6% each.

In some variations, the set of micro-organospheres may be cultured, according to step 932. For example, culture media may be provided to the micro-organospheres to enable them to establish and grow. In some variations, micro-organospheres may be cultured for any desired time, or may be cryopreserved and/or assayed immediately. In some variations, the micro-organospheres may be cultured between about 1 day and about 3 days, between about 1 day and about 4 days, between about 1 day and about 5 days, between about 1 day and about 6 days, between about 1 day and about 7 days, between about 1 day and about 8 days, between about 1 day and about 9 days, between about 1 day and about 10 days, between about 1 day and about 11 days, between about 1 day and about 14 days, including all sub-values and ranges in-between. In some variations, the cells in the micro-organosphere may grow and/or divide (e.g., double) for up to about six passages. After culturing, the cells may be, for example, cryopreserved and/or assayed.

In some variations, culture media for micro-organospheres may contain a basal media (e.g., DMEM F12 or RPMI 1640), a buffer (e.g., HEPES), glutamate, an antibiotic, combinations thereof, and the like. Culture media may further be supplemented with growth factors appropriate to the cell type being cultured. Table 1 provides illustrative growth factors that may be used to supplement growth media to generate organoids for the indicated cell types.

TABLE 1

| Cell Type | Illustrative Growth Factors |
|---|---|
| Colorectal cancer | A83-01, B27, EGF, [Leu15]-Gastrin I, N-Acetylcysteine, |
| | Nicotinamide, Noggin, Primocin, Prostaglandin E2, R-Spondin 1, |
| | SB202190, Y-27632 |
| Small intestine and | A83-01, B27, EGF, [Leu15]-Gastrin I, N-Acetylcysteine, N2, |
| colon | Nicotinamide, Noggin, R-Spondin 1, SB202190, Mouse Recombinant |
| | Wnt-3A, Y-27632 |
| Lung and trachea | A83-01, B27, FGF7, FGF10, N-Acetylcysteine, Nicotinamide, |
| | Noggin, R-Spondin 1, Primocin, SB202190, Y-27632 |
| Breast cancer | A83-01, B27, EGF, FGF7, FGF10, N-Acetylcysteine, Neuregulin I, |
| | Nicotinamide, Noggin, Primocin, R-Spondin 3, SB202190, Y-27632 |
| Esophageal | B27 w/o vitamin A, CultureOne supplement, EGF, FGF10, HGF, N2, |
| | Noggin |
| Liver and spleen | A83-01, B27 (w/o vitamin A), CHIR99021, EGF, FGF7, FGF10, |
| | HGF, N2, N-Acetylcysteine, Nicotinamide, R-Spondin 1, [Leu15]- |
| | Gastrin I, TGFa, Y-27632 |
| Kidney | A83-01, B27, EGF, FGF10, N-Acetylcysteine, Primocin, R-Spondin |
| | 1, Y-27632 |
| Stomach | A83-01, B27 w/o vitamin A, EGF, FGF10, [Leu15]-Gastrin I, N- |
| | Acetylcysteine, Noggin, Primocin, R-Spondin 1, Mouse Recombinant |
| | Wnt-3A, Y-27632 |
| Brainstem and | Neurobasal, 2-mercaptoethanol, B27 w/o vitamin A, Insulin, MEM- |
| cerebral | NEAA, N2 |
| Cardiac | Activin A, B27, BMP-4, CHIR99021, EGF, FGF-2, L-ascorbic acid 2- |
| | phosphate sesquimagnesium salt hydrate |
| Testicular | EGF, Insulin-Transferrin-Selenium |
| Olfactory | B27, EGF, FGF, human, Jagged-1, N2, N-Acetylcysteine, Noggin, R- |
| | Spondin 1, Mouse Recombinant Wnt-3A, Y-27632 |
| Pancreas | A83-01, B27, EGF, FGF10, [Leu15]-Gastrin I, N-Acetylcysteine, |
| | Nicotinamide, Noggin, Primocin, R-Spondin 1, Mouse Recombinant |
| | Wnt-3A |
| Sarcoma | L-Glutamine, Penicillin/Streptomycin, Fetal Bovine Serum, HI |
| Cholangiocarcinoma, | A83-01, B27, EGF, Forskolin, [Leu15]-Gastrin I, N2, N- |
| biliary duct | Acetylcysteine, Nicotinamide, R-Spondin 1, Y-27632 |
| Ovarian | 17-B Estradiol, A83-01, B27 minus Vitamin A, EGF, HGF, IGF1, N2 |
| | Supplement, N-Acetylcysteine, Neuregulin I, Nicotinamide, Noggin, |
| | R-spondin 1, SB203580 (p38i), Y-27632 |
| Liver hepatocellular | A83-01, B27, EGF, FGF10, forskolin, [Leu15]-Gastrin I, HGF, N2, N- |
| carcinoma | Acetylcysteine, Nicotinamide, R-Spondin 1, Mouse Recombinant |
| | Wnt-3A |
| Head and neck | A83-01, B27, CHIR99021, EGF, FGF2, FGF10, forskolin, N- |
| cancer | Acetylcysteine, Nicotinamide, Noggin, Prostaglandin E2, R-Spondin |
| | 1, Y-27632 |
| Liver | Non-Essential Amino Acids, Normacin, A38-01, B27, N2, N- |
| | Acetylcysteine, Nicotinamide, Y-27632, CHIR99021, EGF, HGF, |
| | TNFa, Dexamethasone (DEX) |

In an illustrative method, a tissue sample from a clinical biopsy may be minced or resectioned and then suspended in a temperature-sensitive gel (such as Matrigel®) at about 4° C., and thereafter flowed through a microfluidic droplet chip. In some variations, the number of cells in a homogenized tissue sample may be estimated using an automated cell counter, and then resuspended in gel at a specific desired density in order to provide a predetermined number of cells per droplet based on a predetermined droplet volume. In some variations, a core T-junction of a microfluidic device may be configured to generate gel-based water-in-oil droplets that are substantially uniform in volume and material composition. The homogenized tissue sample in gel may be partitioned into droplet “micro-reactors” and the gel may solidify upon incubation at about 37° C. De-emulsification may recover micro-organosphere containing droplets from the oil phase. The resulting product may comprise, for example, thousands of uniform gel tumor droplets that are compatible with traditional 3D cell culture techniques.

In further illustrative methods, patient/donor-derived micro-organospheres may be periodically monitored using imaging-based approaches for the acquisition of 3D structure (e.g., multiple cells and intercellular contacts) that may more accurately mimic the biology of the parental tumor than 2D culture formats. In some variations, the determination of “establishment” is made based on the systemic acquisition of 3D structure across a large representative sample of droplets based on the imaging data described herein. The imaging data may comprise microscopic images that are taken once daily, and analyzed to estimate the diameter of objects inside the droplets that may be used to generate plots of object size distributions. Upon systemic and consistent growth of droplet objects past the diameter representative of single cells, a sample may be considered as “established,” and thus biologically representative of the parental tumor.

In some variations, organoids within micro-organospheres having measured surface areas of greater than about 700 μm 2 may be considered established. The maximum surface area for an organoid taking up the whole droplet is about 96,000 μm 2. As assay wells may have more than one droplet per well, a well may be considered established and ready for downstream assaying when at least one droplet within the well meets a set of predetermined criteria (e.g., surface area greater than about 700 μm 2). In some variations, assays wells may be considered established when about 30% of the droplets measured in a well have at least one organoid with a surface area of about 700 μm².

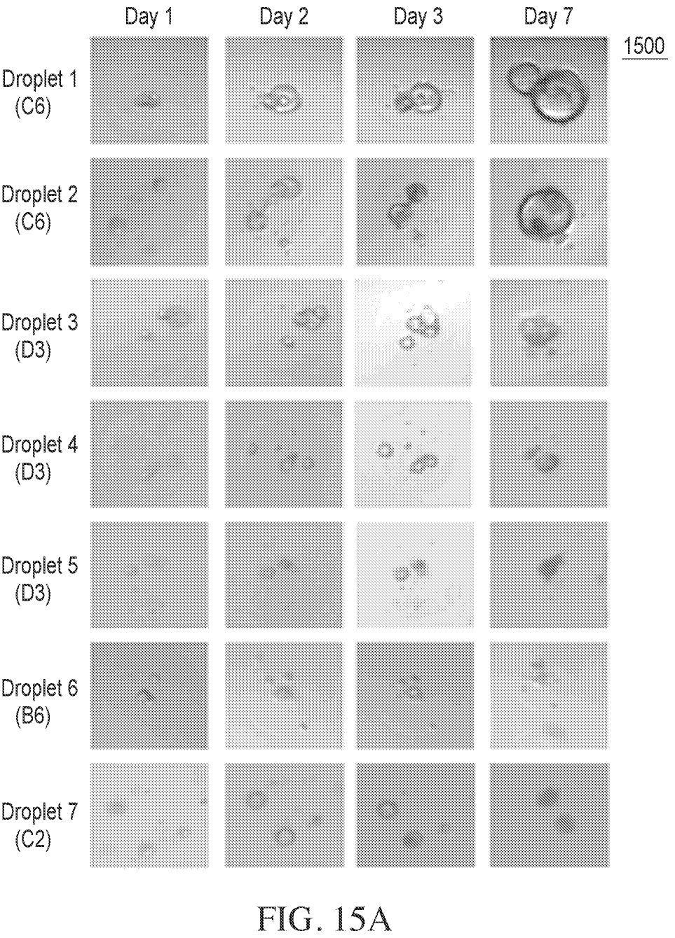

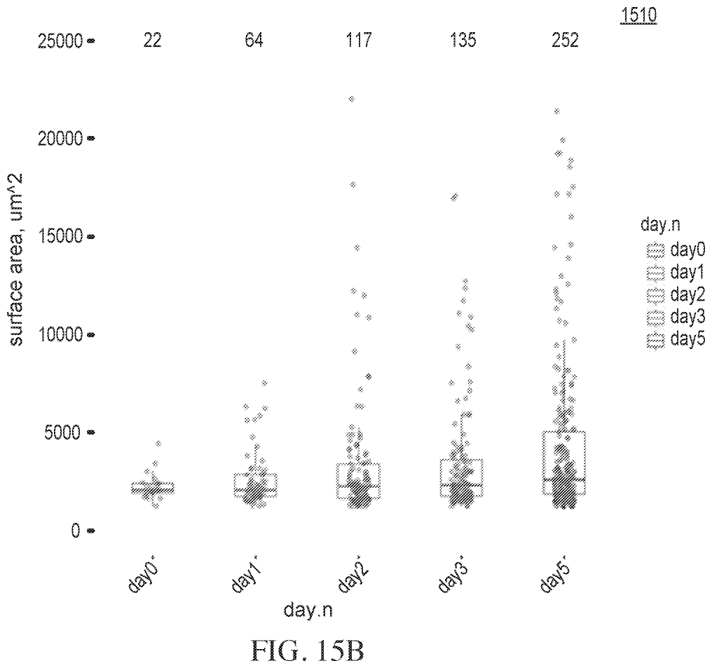

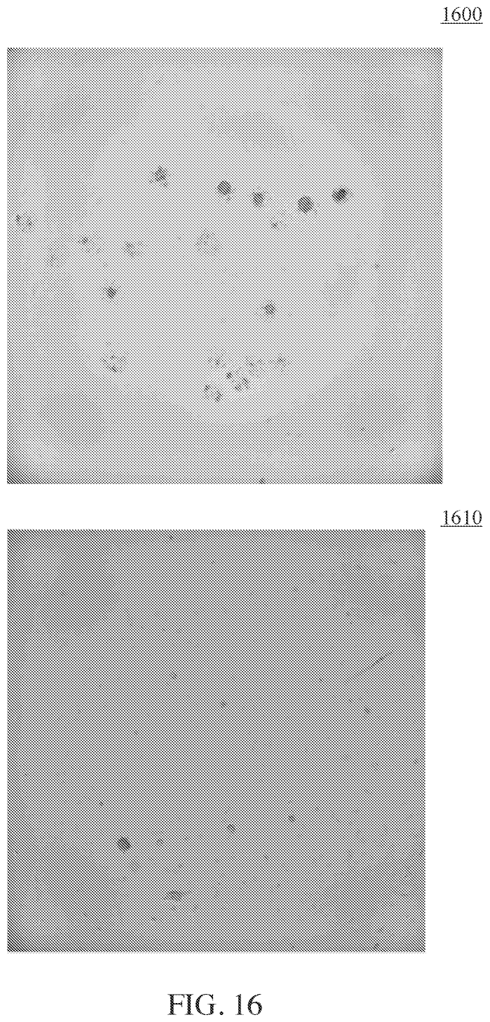

As shown in the images (1500, 1510) of FIGS. 15A and 15B, when cultured, organoids within micro-organospheres rapidly grow and establish themselves as organoids. During the initial growth stages, a single droplet may comprise a plurality of organoids. As time progresses, multiple organoids may merge into a larger organoid and form a single organoid per droplet. The images (1600, 1610) of FIG. 16 allows comparison of micro-organoids to conventional organoids. For example, a fresh clinical breast cancer sample was digested and split in half for micro-organosphere and organoid generation. The micro-organospheres were seeded at 60 cells/droplet in a 96-well plate, and an equivalent number of cells/well were seeded in Matrigel® domes for organoid culture in a separate 96-well plate. After about 3 days, the micro-organospheres had already formed large 3D structures (approximately 200 μm in diameter) and were ready for a drug assay, whereas 3D structures in the conventional organoid cultures were small and sparsely distributed. 4× images of a representative well from each culture are shown in respective images (1600, 1610) of FIG. 16.

The micro-organospheres described herein can be used as healthy tissue models or diseased tissue models. Accordingly, the present disclosure relates to a method of determining a patient's response to a treatment, the method comprising: (a) obtaining a biological sample having cells from the patient; (b) encapsulating the cells in micro-organospheres; (c) contacting the micro-organospheres with the treatment; (d) performing an assay on the micro-organospheres; and (e) determining the response to treatment based on the results from the assay. In some variations, the treatment includes a drug or a drug candidate. In some variations, the treatment includes a chemotherapeutic, targeted, or immune cell-based therapy. Notably, the present disclosure allows rapid assessment of a patient's response to a treatment, e.g., within about 14 days of receiving cells from the patient. In some variations, the assessment may include determining a response of healthy tissue to a predetermined treatment (e.g., drug or drug candidate). In some variations, the response may include toxicity and/or another measurable drug response.

In some variations, the assay is a cell viability assay. Examples of cell viability assays include, but are not limited to, cellTiter-Glo®, cellTiter-Glo® 3D, live/death fluorescent labels, and imaging.

In some variations, the assay is a cell painting assay, e.g., fluorescent staining of cells in-situ in micro-organospheres. In cell painting assays, one or more fluorophores are tagged to one or more protein/cellular or extra-cellular structure. For example, see Bray et al., “Cell Painting, a high-content image-based assay for morphological profiling using multiplexed fluorescent dyes,” Nature Protocols 2016, 11, 1757-1774, the contents of which are incorporated by reference.

Additional data can also be obtained from the biological sample and/or micro-organospheres by performing various characterization methods known in the art, such as histology (e.g., E&H and IHC staining of FFPE blocks of DMOS), DNA/RNA testing, bulk cell viability assays (e.g., cellTiter-Glo®/cellTiter-Glo® 3D), proteomics, and ctDNA assay from supernatant. The characterization methods can be performed on each of the micro-organospheres as a whole or a portion thereof, such as cells or microstructures in the micro-organospheres. Cells can be extracted from micro-organospheres to be analyzed or manipulated independently, for example through single cell sequencing, flow cytometry, FACS, or other techniques. The characterization methods can also be performed on the supernatant obtained from a solution or suspension having the micro-organospheres.

In some variations, the biological sample is a tumor tissue. As such, in addition to the micro-organospheres, measurements performed on the tumor tissue itself can provide additional data to help determine a patient's response to a treatment.

As used herein, sterile should be understood as a non-limiting description of some variations, an optional feature providing advantages in operation of certain systems and methods of the disclosure. Maintaining sterility is typically desirable for cell processing but may be achieved in various ways, including but not limited to providing sterile reagents, media, cells, and other solutions; sterilizing cartridge(s) and/or cartridge component(s) after loading (preserving the cell product from destruction); and/or operating the system in a sterile enclosure, environment, building, room, or the like. Such user or system performed sterilization steps may make the cartridge or cartridge components sterile and/or preserve the sterility of the cartridge or cartridge components.

All references cited are herein incorporated by reference in their entirety.

As used herein, the singular forms “a”, “an” and “the” include plural referents unless the context clearly dictates otherwise. “And” as used herein is interchangeably used with “or” unless expressly stated otherwise.

As used herein, the terms “substantially,” “approximately,” and “about” generally mean plus or minus 10% of the value stated, e.g., about 100 would include 90 to 110.

As used herein, the phrase “and/or” should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” may refer, in one variation, to A only (optionally including elements other than B); in another variation, to B only (optionally including elements other than A); in yet another variation, to both A and B (optionally including other elements); etc.

As used herein, the term “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of” or “exactly one of” “Consisting essentially of” when used in the claims, shall have its ordinary meaning as used in the field of patent law.

As used herein, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) may refer, in one variation, to at least one, optionally including more than one, A, with no B present (and optionally including elements other than B); in another variation, to at least one, optionally including more than one, B, with no A present (and optionally including elements other than A); in yet another variation, to at least one, optionally including more than one, A, and at least one, optionally including more than one, B (and optionally including other elements); etc.

All variations of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” “below,” and words of similar import, when used in this application, shall refer to this application as a whole and not to any particular portions of the application.

While variations of the present invention have been shown and described herein, those skilled in the art will understand that such variations are provided by way of example only. Numerous variations, changes, and substitutions will now occur to those skilled in the art without departing from the invention. It should be understood that various alternatives to the variations of the invention described herein may be employed in practicing the invention. It is intended that the following claims define the scope of the invention and that methods and structures within the scope of these claims and their equivalents be covered thereby.

What is claimed herein is:

1. A system, comprising:
a micro-organosphere generator comprising a microfluidic device and configured to form a set of micro-organospheres from a mixture of a biological sample and a fluid; and
a controller coupled to an imaging device, the controller configured to:
receive imaging data corresponding to one or more of the mixture or the set of micro-organospheres; and
estimate one or more characteristics of the set of micro-organospheres based at least on the imaging data.

2. The system of claim 1, further comprising:
an imaging device configured to generate the imaging data corresponding to the one or more of the mixture or the set of micro-organospheres.

3. The system of claim 2, further comprising:
a cell culture vessel coupled to the imaging device and configured to culture the set of micro-organospheres in a plurality of wells, and
the controller further configured to:
estimate a number of micro-organospheres in the plurality of wells based at least on the imaging data.

4. The system as in any of the preceding claims, further comprising:
one or more sensors coupled to the microfluidic device and configured to generate sensor data corresponding to the mixture or the set of micro-organospheres, and
the controller further configured to:
receive the sensor data from the one or more sensors; and
estimate one or more characteristics of the set of micro-organospheres based at least on the sensor data.

5. The system of claim 4, further comprising:
one or more pumps coupled to the microfluidic device and configured to control fluid flow to the microfluidic device; and
a temperature regulator coupled to the microfluidic device, sample source, or fluid source, and configured to control a temperature of the sample source, the fluid source, the mixture, or the set of micro-organospheres, and
the controller configured to:
modify one or more of the pump or the temperature based at least on the imaging data and the sensor data.

6. The system as in any of the preceding claims, further comprising:
a polymerizer fluidically coupled to the microfluidic device and configured to polymerize the mixture to form the set of micro-organospheres.

7. The system as in any of the preceding claims, further comprising:
a demulsifier fluidically coupled to the microfluidic device and configured to demulsify the mixture to form the set of micro-organospheres.

8. The system as in any of the preceding claims, further comprising:
an agitator configured to agitate the micro-organospheres within a fluid at a predetermined concentration.

9. The system as in any of the preceding claims, wherein the one or more of the characteristics of the set of micro-organospheres comprises one or more of a micro-organosphere diameter, a total number of cells, or a number of living cells.

10. The system as in any of the preceding claims, wherein the controller is configured to estimate one or more characteristics of the mixture based at least on the imaging data.

11. The system of claim 10, wherein the one or more of the characteristics of the mixture comprises a total number of cells and a number of living cells.

12. The system as in any of the preceding claims, wherein the imaging data corresponds to the biological sample, and the controller is configured to estimate one or more characteristics of the biological sample based at least on the imaging data.

13. The system of claim 12, wherein the one or more of the characteristics of the biological sample comprises a total number of cells and a number of living cells.

14. The system of claim 7, wherein the demulsifier comprises a flow separator configured to isolate the set of micro-organospheres.

15. The system of claim 14, wherein the flow separator extends along a length of the demulsifier.

16. The system as in any of the preceding claims, wherein the set of micro-organospheres comprises a diameter of between about 200 μm and about 400 μm.

17. The system as in any of the preceding claims, wherein the micro-organosphere generator is configured to form the set of micro-organospheres from the biological sample comprising a volume of up to about 1 mL.

18. The system as in any of the preceding claims, wherein the micro-organosphere generator is configured to form the set of micro-organospheres from the biological sample comprising less than about 10,000 cells.

19. The system of claim 18, wherein the biological sample comprises between about 3,500 cells and about 7,500 cells.

20. The system as in any of the preceding claims, wherein the micro-organosphere generator is configured to form the set of micro-organospheres from the biological sample having a volume of about 5 μL to about 5 mL.

21. The system of claim 20, wherein the biological sample has a volume of about 5 μL, about 10 μL, about 20 μL, about 35.3 μL, about 50 μL, about 100 μL, about 250 μL about 500 μL, about 1 mL, about 1.5 mL, about 2 mL, about 2.5 mL, about 3 mL, about 3.5 mL, about 4 mL, about 4.5 mL, or about 5 mL.

22. The system as in any of the preceding claims, wherein the set of micro-organospheres comprises a set of non-cellular objects.

23. The system of claim 22, wherein the set of non-cellular objects comprise one or more inert particles.

24. The system of claim 23, wherein the set of non-cellular objects comprises between about 1 inert particle and about 5,000 inert particles.

25. A system, comprising:
a micro-organosphere generator configured to form a set of micro-organospheres from a mixture of a biological sample and a fluid; and
a controller configured to:
receive imaging data corresponding to the set of micro-organospheres; and
identify the set of micro-organospheres comprising a diameter of between about 50 μm and about 500 μm based at least on the imaging data.

26. The system as in any of the preceding claims, further comprising:
an imaging device configured to generate the imaging data corresponding to the set of micro-organospheres.

27. The system as in any of the preceding claims, wherein the biological sample corresponds to a patient biopsy.

28. A method of making a micro-organosphere composition in a system according to any one of claims 1 to 27, comprising:
providing the biological sample comprising dissociated cells and an unpolymerized base material;
forming the mixture from the biological sample in an immiscible solution; and
polymerizing the mixture to form a set of micro-organospheres.

29. The method as in any of the preceding claims, further comprising dissociating the biological sample to obtain the dissociated cells.

30. The method as in any of the preceding claims, wherein the base material is temperature sensitive and polymerization occurs when the temperature of the mixture is increased.

31. The method as in any of the preceding claims, wherein the set of micro-organospheres comprise a mean diameter of between about 50 μm and about 500 μm with a coefficient of variability (CV) less than about 30% CV, less than about 20% CV, or less than about 10% CV.

32. The method as in any of the preceding claims, further comprising:
sorting the organospheres by size to form the set of micro-organospheres comprising a mean diameter of between about 50 μm and about 500 μm with a coefficient of variability (CV) less than about 30% CV, less than about 20% CV, or less than about 10% CV; or
controlling one or more flow rates within the micro-organosphere generator to form the set of micro-organospheres comprising a mean diameter of between about 50 μm and about 500 μm with a coefficient of variability (CV) less than about 30% CV, less than about 20% CV, or less than about 10% CV.

33. The method as in any of the preceding claims, further comprising performing an assay on the micro-organospheres to determine treatment response.

34. The method of claim 33, wherein the assay is a cell viability assay or a cell painting assay.

35. The method of claim 33, wherein the assay is performed in 14 days or less from when the biological sample is obtained from a patient.

36. The method as in any of the preceding claims, wherein the micro-organospheres comprise between about 1 dissociated primary cell and about 1,000 dissociated primary cells distributed within the base material.

37. The method as in any of the preceding claims, wherein the biological sample corresponds to a patient biopsy.

38. A micro-organosphere composition comprising: a plurality of micro-organospheres with each micro-organosphere including a base material and at least one organoid, wherein the plurality of micro-organospheres comprise parameters comprising a predetermined number of cells per droplet, a predetermined number of droplets in the composition, and/or a predetermined droplet size, wherein each of the parameters independently comprise a coefficient of variability (CV) less than about 30% CV, less than about 20% CV, or less than about 10% CV.

39. The composition as in any of the preceding claims, wherein the mean diameter of each micro-organosphere in the composition is between about 50 μm and about 500 μm.

40. The composition of claim 39, wherein the mean diameter of each micro-organosphere in the composition comprises a coefficient of variability (CV) of less than about 30% CV, less than about 20% CV, or less than about 10% CV.

41. The composition as in any of the preceding claims, wherein each micro-organosphere comprises a base material and only one organoid.

42. The composition as in any of the preceding claims, wherein each micro-organosphere further comprises an inert particle.

43. The composition of claim 42, wherein the inert particle is a magnetic particle, a magnetizable particle, a fluorescent particle, or a combination thereof.

44. The composition of claim 42, wherein each micro-organosphere comprises between about 1 inert particle and about 5,000 inert particles.

45. The composition of any of the preceding claims, wherein the plurality of micro-organospheres comprise tissue from a patient biopsy.

46. The composition of claim 45, wherein the tissue comprises non-cultured cells.

47. The method as in any of the preceding claims, wherein the micro-organospheres comprise between about 1 dissociated primary cell and about 1,000 dissociated primary cells distributed within the base material.

48. A method of immobilizing micro-organospheres in a well or culture plate, the method comprising:
providing a plurality of micro-organospheres, each micro-organosphere comprising a base material, at least one organoid, and a magnetic or magnetizable particle, and
applying a magnetic field to the well or culture plate, thereby immobilizing the micro-organospheres to a surface of the well or culture plate.

49. The method as in any of the preceding claims, wherein:
the well or the culture plate has a bottom; and
the micro-organospheres are immobilized to the bottom of the well or culture plate.

50. A method of immobilizing micro-organospheres in a well or culture plate that has a bottom, the method comprising:
providing a plurality of micro-organospheres, each micro-organosphere comprising a base material and at least one organoid;
functionalizing the bottom with an antibody that binds the base material; and
contacting the micro-organospheres with the antibody, thereby immobilizing the micro-organospheres to the bottom.

51. The method as in any of the preceding claims, wherein the antibody is immobilized on the bottom by incubation.

52. The method as in any of the preceding claims, wherein the bottom is coated with protein A and/or protein G prior to the functionalization.

53. A method of determining a patient's response to a treatment, the method comprising:
performing an assay on micro-organospheres, wherein the micro-organospheres are produced by:
mixing a biological sample comprising dissociated cells from the patient with an unpolymerized base material in an immiscible solution to produce a mixture; and
polymerizing the mixture to form a set of micro-organospheres.

54. The method as in any of the preceding claims, wherein the assay is a cell viability assay or a cell painting assay.

55. The method as in any of the preceding claims, wherein the assay is performed in about 14 days or less from when the biological sample is obtained from a patient.

56. The method as in any of the preceding claims, wherein the micro-organospheres comprise between about 1 dissociated primary cell and about 1,000 dissociated primary cells distributed within the base material.
